(19) United States
(12) Patent Application Publication
(10) Pub. No.: US 2025/0032430 A1
(43) Pub. Date: Jan. 30, 2025

(54) USES OF RHODOQUINONE FOR THE TREATMENT OF DISEASE

(71) Applicants: Whitehead Institute for Biomedical Research, Cambridge, MA (US); University of Massachusetts, Boston, MA (US)

(72) Inventors: David M. Sabatini, Cambridge, MA (US); Jessica Spinelli, Worcester, MA (US)

(73) Assignees: Whitehead Institute for Biomedical Research, Cambridge, MA (US); University of Massachusetts, Boston, MA (US)

(21) Appl. No.: 18/706,551

(22) PCT Filed: Nov. 2, 2022
(86) PCT No.: PCT/US2022/079177

§ 371 Date: May 1, 2024

Related U.S. Application Data
(60) Provisional application No. 63/274,880, filed on Nov. 2, 2021.

(51) Int. Cl. A61K 31/136; A61K 45/06

(52) U.S. Cl. CPC A61K 31/136; A61K 45/06

(57) ABSTRACT

Provided herein are compounds of Formulae (I) and (II), and pharmaceutically acceptable salts, solvates, hydrates, polymorphs, co-crystals, tautomers, stereoisomers, isotopically labeled derivatives, and prodrugs thereof. Also provided are methods, uses, and kits involving the disclosed compounds and pharmaceutical compositions thereof for treating and/or preventing a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject.

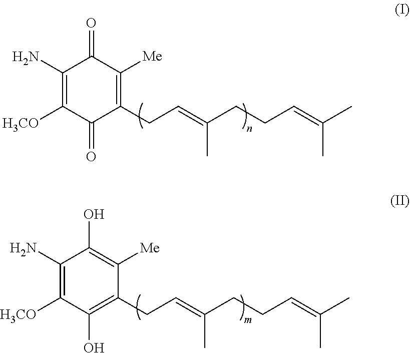

### RELATED APPLICATIONS

The present application claims priority under 35 U.S.C. § 119(e) to U.S. provisional application, U.S. Ser. No. 63/274,880, filed Nov. 2, 2021, the entire contents of which are incorporated herein by reference.

### BACKGROUND OF THE INVENTION

The flow of electrons through the mitochondrial electron transport chain (ETC.) supports a diverse set of cellular processes, such as the synthesis of metabolites that support macromolecule production as well as the regulation of signaling and cell death pathways. Electrons enter the ETC through many routes, including from complex I and dihydroorotate dehydrogenase (DHODH), move between complexes via the electron carrier ubiquinol (QH2), and exit by reducing a terminal electron acceptor. In mammalian cells, the canonical view is that oxygen serves as the sole terminal electron acceptor, and that its reduction is necessary for the reoxidation of ubiquinol (QH2) into ubiquinone (Q) and thus the continuous input of electrons into the ETC. However, under a variety of physiological states mammalian cells can exist in hypoxic niches while maintaining functions that require the flow of electrons into the ETC, including de novo pyrimidine biosynthesis and NADH oxidation.

### SUMMARY OF THE INVENTION

This disclosure is based in part on the discovery that mammalian cells possess a novel metabolite, rhodoquinone-9 and rhodoquinone-10 (shown below), which is analogous to ubiquinone-9 and ubiquinone-10 in its ability to carry electrons in the mitochondrial electron transport chain.

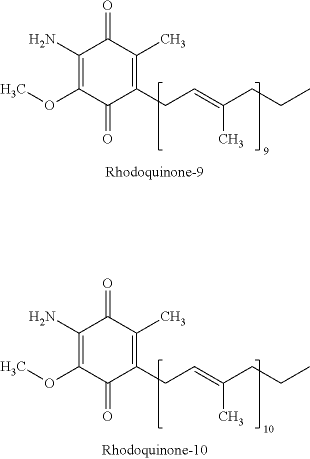

Rhodoquinone is not present in cells cultured in a petri dish, only in tissues isolated from an organism. In addition, it was discovered that rhodoquinone has the ability to transfer electrons to fumarate in the mammalian electron transport chain, and fumarate reduction sustains mitochondrial functions in hypoxia. Thus, rhodoquinones or rhodoquinols and their pharmaceutically acceptable salts, solvates, hydrates, polymorphs, co-crystals, tautomers, stereoisomers, isotopically labeled derivatives, or prodrugs thereof can be used as a therapeutic to treat a variety diseases. For example, rhodoquinone and/or rhodoquinol may effectively treat metabolic disorders (e.g., obesity, diabetes), hypoxia related diseases, i.e., where the body or any region of the body has been deprived of adequate oxygen supply (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder).

Described herein are methods of treating metabolic disorders (e.g., obesity, diabetes), hypoxia related diseases (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder) in a subject in need thereof, the method comprising administering to the subject a therapeutically effective amount of a compound of Formula (I):

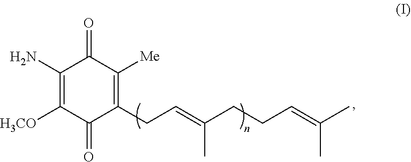

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof.

Also described herein are methods of treating metabolic disorders (e.g., obesity, diabetes), hypoxia related diseases (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder) in a subject in need thereof, the method comprising administering to the subject a therapeutically effective amount of a compound of Formula (II):

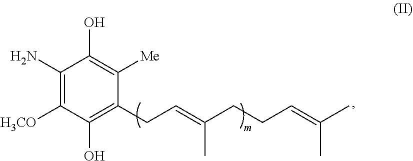

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof.

Further described herein are methods of using the compounds of Formula (I) and Formula (II), or a pharmaceutically acceptable salt, solvate, hydrate, polymorph, co-crystal, tautomer, stereoisomer, isotopically labeled derivative, or prodrug thereof, to study the mammalian electron transport chain. The compounds described herein may be useful in treating and/or preventing metabolic disorders (e.g., obesity, diabetes), hypoxia related diseases (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder).

In another aspect, the present disclosure provides pharmaceutical compositions including a compound of Formula (I) or Formula (II) as described herein, and optionally a pharmaceutically acceptable excipient. In certain embodiments, the pharmaceutical compositions described herein include a therapeutically or prophylactically effective amount of a compound described herein. The pharmaceutical composition may be useful for treating and/or preventing metabolic disorders (e.g., obesity, diabetes), hypoxia related diseases (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder) in a subject in need thereof.

In another aspect, described herein are methods for treating and/or preventing metabolic disorders (e.g., obesity, diabetes), hypoxia related diseases (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder) using a compound described herein, which may be optionally administered in combination with an additional pharmaceutical agent, for example, a vitamin, an antioxidant, an anti-inflammatory, an anti-cancer agent, an anti-obesity agent, a probiotic, an antibiotic, a statin, or a plasmid (e.g., a plasmid encoding a protein (e.g., an enzyme enabling in vivo conversion of ubiquinone to rhodoquinone (e.g., RquA))). In certain embodiments, the additional pharmaceutical agent is an anti-obesity agent, a probiotic, an antibiotic, a statin, or a plasmid (e.g., a plasmid encoding a protein (e.g., an enzyme enabling in vivo conversion of ubiquinone to rhodoquinone (e.g., RquA))).

In another aspect, described herein are methods for activating the electron transport chain using a compound described herein, which may be optionally administered in combination with an additional pharmaceutical agent, for example, a vitamin, an antioxidant, an anti-inflammatory, an anti-cancer agent, an anti-obesity agent, a probiotic, an antibiotic, a statin, or a plasmid (e.g., a plasmid encoding a protein (e.g., an enzyme enabling in vivo conversion of ubiquinone to rhodoquinone (e.g., RquA))). In certain embodiments, the additional pharmaceutical agent is an anti-obesity agent, a probiotic, an antibiotic, a statin, or a plasmid (e.g., a plasmid encoding a protein (e.g., an enzyme enabling in vivo conversion of ubiquinone to rhodoquinone (e.g., RquA))).

In yet another aspect, the present disclosure provides compounds of Formula (I) and Formula (II), and pharmaceutically acceptable salts, solvates, hydrates, polymorphs, co-crystals, tautomers, stereoisomers, isotopically labeled derivatives, or prodrugs thereof, which may be optionally administered in combination with an additional pharmaceutical agent for use in the treatment of metabolic disorders (e.g., obesity, diabetes), hypoxia related diseases (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder) in a subject.

Another aspect of the present disclosure relates to kits comprising a container with a compound of Formula (I) or Formula (II), or a pharmaceutical composition thereof, as described herein. The kits described herein may include a single dose or multiple doses of the compound or pharmaceutical composition. The kits may be useful in a method of the disclosure. In certain embodiments, the kit further includes instructions for using the compound or pharmaceutical composition. A kit described herein may also include information (e.g., prescribing information) as required by a regulatory agency, such as the U.S. Food and Drug Administration (FDA).

The details of one or more embodiments of the invention are set forth herein. Other features, objects, and advantages of the invention will be apparent from the Detailed Description, Examples, Figures, and Claims.

### Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) or (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formulae (I) and (II), which have cleavable groups and become by solvolysis or under physiological conditions the compounds of Formulae (I) and (II) which are pharmaceutically active in vivo. Such examples include, but are not limited to, ester derivatives, amide derivatives, and the like. Other derivatives of the compounds of this invention have activity in both their acid and acid derivative forms, but in the acid sensitive form often offers advantages of solubility, tissue compatibility, or delayed release in the mammalian organism (see Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of Formulae (I) and (II) are particular prodrugs.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. A non-human animal may be a transgenic animal.

The terms “administer,” “administering,” or “administration” refer to implanting, absorbing, ingesting, injecting, inhaling, or otherwise introducing an inventive compound or a pharmaceutical composition thereof.

The terms “treatment,” “treat,” and “treating” refer to reversing, alleviating, delaying the onset of, or inhibiting the progress of a “pathological condition” (e.g., metabolic disorders (e.g., obesity, diabetes), hypoxia related diseases (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder), or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “prevent,” “preventing,” or “prevention” refers to a prophylactic treatment of a subject who does not have and did not have a disease but is at risk of developing the disease or is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound of Formula (I) or Formula (II) refers to an amount sufficient to elicit the desired biological response, i.e., activating the electron transport chain in a subject, tissue, or cell, or treating the condition, for example, treating a proliferative disease, inflammatory disease, metabolic disorder, neuromuscular disorder, neurodegenerative disorder, hypoxia, ischemia, oxidative stress, or a mitochondrial DNA related disorder. As will be appreciated by those of ordinary skill in this art, the effective amount of a compound of Formula (I) or Formula (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) or Formula (II) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain. In certain embodiments, the tissue is an organ. In certain embodiments, the organ is an ex vivo organ (e.g., an organ being transported and/or stored for organ transplant).

The term “hypoxia” or “hypoxic condition” as used herein refers to a state or condition in which oxygen in a subject or in one or more tissues of a mammal is below physiologic levels, e.g., is at a less than optimal level. In some embodiments, hypoxia may result from stress such as aerobic exercise, physical weight pressure, anesthesia, surgery, anemia, acute respiratory distress syndrome, chronic illness, chronic fatigue syndrome, trauma, burns, skin ulcers, cachexia due to cancer and other catabolic states and the like. In certain embodiments, hypoxia may result from a complete or partial blockage of a blood vessel, abnormally low blood pressure, vasoconstriction, reduced air flow in and out of the lungs, or damaged lung tissue. In certain embodiments, the hypoxia can be caused by a stroke, myocardial infarction, heart failure, or trauma.

In some embodiments, hypoxia is or comprises “ischemia” or “ischemic conditions” in which tissues are oxygen-deprived due to reduction in blood flow, as due to constriction in, or blockage of, a blood vessel. Ischemia or ischemic conditions include those caused by coronary artery disease, cardiomyopathy, including alcoholic cardiomyopathy, angioplasty, stenting, heart surgery such as bypass surgery or heart repair surgery (“open-heart surgery”), organ transplantation, prolonged weight pressure on tissues (pressure ulcers or bedsores), ischemia-reperfusion injury which can cause damage to transplanted organs or tissue, and the like.

In some embodiments, treatments described herein may, for example, increase energy level, strength and/or well-being of a subject suffering from hypoxia even if they do not treat one or more aspects of an underlying condition (e.g., viral or bacterial infection, exposure to bacterial or other toxins, low red-cell counts, aging, cancer, continued exercise, high altitude exercise).

In some embodiments, the terms “hypoxia” or “hypoxic condition” refer to a condition of low oxygen content in the blood. In some embodiments, hypoxia or hypoxic conditions may be defined by arterial PO2 values less than approximately 80 mm Hg and venous PO2 values less than approximately 30 mm Hg. In some embodiments, hypoxia or hypoxic conditions may be defined by arterial PO2 values less than approximately 60 mm Hg. In certain embodiments, hypoxia or hypoxic conditions may be defined by arterial PO2 values less than approximately 50 mm Hg. In a particular embodiment, hypoxia or hypoxic conditions may be defined by arterial PO2 values between approximately 50-20 mm Hg. In some embodiments, hypoxia or hypoxic conditions may be defined by intra-tissue PO2 levels less than about 10 mm Hg. In some embodiments, hypoxia or hypoxic conditions may be defined by intra-tissue PO2 levels less than about 5 mm Hg. In some embodiments, hypoxia or hypoxic conditions may be defined by intra-tumor PO2 levels less than about 10 mm Hg. In some embodiments, hypoxia or hypoxic conditions may be defined by intra-tumor PO2 levels less than about 5 mm Hg. Hypoxia or hypoxic conditions may be chronic or acute.

Chronic hypoxia, as used herein, may refer to sustained hypoxic conditions that result in a measurable increase in 2HG production. That is, chronic hypoxia may be defined as hypoxic conditions of sufficient duration to allow 2HG to accumulate above baseline levels. In some embodiments, chronic hypoxia is a hypoxic condition of more than 30 minutes, more than 1 hour, more than 2 hours, more than 3 hours, more than 4 hours, more than 5 hours, more than 10 hours, more than 12 hours, more than 24 hours, more than a day, or a week or more in duration. In some embodiments, chronic hypoxia is caused by consumption and depletion of oxygen by tissues or tumor cells between blood capillaries and the hypoxic regions. In contrast to the sustained conditions of chronic hypoxia, acute hypoxia is transient. In some embodiments, acute hypoxia occurs when there a temporary shutdown of vessels or microvasculature in tissues or tumors. In some embodiments, acute hypoxia occurs as a result of fluctuations in red blood cell levels.

A “hypoxia related disease” is a disease, disorder, or condition associated with (e.g., whose incidence or severity correlates with and/or that is characterized by one or more aspects of) hypoxia. In some particular embodiments, a hypoxia related disease, disorder or condition may be or comprise, for example, septic shock, ischemic stroke, myocardial infarction, anemia, pulmonary disease, airway obstruction, acute respiratory distress syndrome, pneumonia, pneumothorax, emphysema, congenital heart defects, atherosclerosis, thrombosis, pulmonary embolism, pulmonary edema, asthma, cystic fibrosis, cancer, certain surgical procedures. In some embodiments, a hypoxia related disease, disorder or condition is associated with stress such as aerobic exercise, physical weight pressure, anesthesia, surgery, anemia, acute respiratory distress syndrome, chronic illness, chronic fatigue syndrome, trauma, burns, skin ulcers, cachexia due to cancer and other catabolic states and the like. In some embodiments, a hypoxia-related disease, disorder or condition is or comprises “ischemia” or “ischemic conditions” in which tissues are oxygen-deprived due to reduction in blood flow, as due to constriction in, or blockage of, a blood vessel. Ischemia or ischemic conditions include those caused by coronary artery disease, cardiomyopathy, including alcoholic cardiomyopathy, angioplasty, stenting, heart surgery such as bypass surgery or heart repair surgery (“open-heart surgery”), organ transplantation, prolonged weight pressure on tissues (pressure ulcers or bedsores), ischemia-reperfusion injury which can cause damage to transplanted organs or tissue, and the like.

Without wishing to be bound by any particular theory, unwanted depletion of bacteria that produce this rhodoquinone and/or rhodoquinol may have severe implications for mitochondrial function in organs that rely on rhodoquinone as an electron carrier. Thus, the loss of this metabolite after antibiotic treatment may affect patient risk for diseases related to ischemia, and supplementation with rhodoquinone and/or rhodoquinol may protect those subjects.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes, such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, lymphoma, non-Hodgkin's lymphoma, leukemia, sarcoma, lung cancer, thyroid cancer, breast cancer, liver cancer, pancreatic cancer, gastric cancer, ovarian cancer, colon cancer, colorectal cancer, skin cancer, esophageal cancer, and carcinoma. Exemplary proliferative diseases include cancers (i.e., “malignant neoplasms,”, sarcoma, lung cancer, thyroid cancer, breast cancer, liver cancer, pancreatic cancer, gastric cancer, ovarian cancer, colon cancer, colorectal cancer, skin cancer, esophageal cancer; carcinoma), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary original tumor to another organ or tissue and is typically identifiable by the presence of a “secondary tumor” or “secondary cell mass” of the tissue type of the primary original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), MYD88-mutated Waldenström's macroglobulinemia, activated B-cell (ABC) diffuse large B-cell lymphoma, mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatic (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation. In certain embodiments, the inflammatory disorder is fibrosis, and the fibrosis is idiopathic pulmonary fibrosis, liver cirrhosis, cystic fibrosis, systemic sclerosis, progressive kidney disease, or cardiovascular fibrosis.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

### BRIEF DESCRIPTION OF THE DRAWINGS

FIG. 1 shows that cells deficient in O₂ reduction retain the capacity to input electrons into the ETC. A. Schematic depicting the electron transport chain (ETC) and the deposition of electrons onto a terminal electron acceptor. B. DHODH activity as determined by stable isotope tracing with 10 mM ¹³C₄-aspartate, which generates ¹³C₃-UTP if DHODH is active. Tracing was performed for 8 hours in 143B cells treated with vehicle (DMSO), 500 nM antimycin, 2 μM brequinar, or in combination; or cultured in 1% O₂ for 24 hours (mean+/−SEM, N=3 per condition). C. Immunoblot analyses of mitochondrial proteins in wild-type (WT), UQCRC2 (complex III) KO, and COX4 (complex IV) KO 143B cells. D. O₂ consumption rate (OCR) of WT, UQCRC2 KO, and COX4 KO 143B cells treated with DMSO or 500 nM antimycin for 1 hour (mean+/−SEM, N=3 per condition). E. DHODH activity as determined using stable isotope tracing of 10 mM ¹³C₄-aspartate, which generates ¹³C₃-UTP if DHODH is active. Tracing was performed for 8 hours in WT, UQCRC2 KO, and COX4 KO 143B cells treated with DMSO or 2 μM brequinar (mean+/−SEM, N=3 per condition). F. Schematic depicting the complex I activity assay on purified mitochondria. NADH initiates the reaction and the absorbance (A₆₀₀) of the oxidized electron acceptor DCPIP is measured over time. G. Complex I activity in mitochondria purified from WT, UQCRC2 KO, and COX4 KO 143B cells in the presence or absence of 1 μM rotenone (complex I inhibitor). P values were calculated using an extra sum of squares F test in GraphPad Prism. H. Polar metabolite profiling of 143B cells treated with DMSO versus 500 nM antimycin for 8 hours, grown in 21% versus 1% O₂, WT versus UQCRC2 KO 143B cells, or WT versus COX4 KO 143B cells, N=3 per condition. For all experiments * indicates P<0.05. Unless otherwise stated, P values were calculated using an unpaired parametric t test.

FIG. 2 shows fumarate accepts electrons via net-reversal of the SDH complex, upon inhibition of O₂ reduction. A. Schematic depicting the question “what is the fate of electrons in the ETC when O₂ cannot be reduced?” B. Schematic showing the expected isotopologues produced during ¹³C₄-aspartate tracing if succinate is generated from fumarate. C. Percent labeled fumarate and succinate from a stable isotope tracing experiment using 3 mM ¹³C₄-aspartate. WT 143B cells were treated with DMSO or 500 nM antimycin for the indicated times (mean+/−SEM, N=3 per timepoint). D. Schematic depicting the reduction of fumarate from either electron leakage onto fumarate or net-reversal of SDH upon antimycin treatment. E. Schematic demonstrating the expected isotopologues of TCA cycle metabolites produced during ¹³C₅¹⁵N₂-glutamine tracing. The forward direction of the SDH reaction can be monitored with the ratio of % labeled fumarate M+4 to % labeled succinate M+4. The reverse direction of the SDH reaction can be monitored with the ratio of % labeled succinate M+3 to % labeled fumarate M+3. F. Fumarate reduction and succinate oxidation as determined using stable isotope tracing of 2 mM ¹³C₅¹⁵N₂-glutamine. Tracing was performed for 8 hours in WT, COX4 KO, and COX4 KO 143B cells expressing the COX4 cDNA and treated with DMSO or 100 nM antimycin for 8 hours. (mean+/−SEM, N=3 per condition). G. Immunoblot analyses for indicated proteins in SDHB KO and SDHB cDNA addback 143B cells. H. Fumarate reduction and succinate oxidation as determined using stable isotope tracing of 2 mM ¹³C₅¹⁵N₂-glutamine. Tracing was performed for 8 hours in WT, SDHB KO, and SDHB KO 143B cells expressing the SDHB cDNA and treated with DMSO or 100 nM antimycin for 8 hours (mean+/−SEM, N=3 per condition). I. SDH activity in purified mitochondria from WT and SDHB KO 143B cells. The succinate oxidation reaction was initiated by adding 10 mM succinate and monitored by the production of fumarate over time. The fumarate reduction reaction was initiated with 10 mM fumarate and 1 mM NADH and monitored via the production of succinate over time. 1 μM antimycin and 1 μM piericidin were included as indicated (mean+/−SEM, N=3 per timepoint). Data points were fitted using linear regression. For all experiments * indicates P<0.05. P values were calculated using an unpaired parametric t test.

FIG. 3 shows fumarate reduction is required to maintain nucleotide biosynthesis and the mitochondrial membrane potential in cells deficient in O₂ reduction. A. Schematic depicting the potential impact of alternative oxidase (AOX) on the accumulation of QH₂ in cells deficient for complex III or IV activity and the consequences for fumarate reduction. B. Ratio of ion counts of ubiquinol to ubiquinone as measured by LC-MS on mitochondria isolated from WT and SDHB KO 143B cells expressing or not AOX and treated with DMSO or 500 nM antimycin for 3 hours (mean+/−SEM, N=4 per condition). C. Relative fumarate reduction as determined using stable isotope tracing of 2 mM ¹³C₅¹⁵N₂-glutamine and The ratio of % Succinate M+3 to % Fumarate M+3, representing fumarate reduction in a stable isotope tracing experiment using 2 mM ¹³C₅¹⁵N₂-glutamine. Tracing was performed for 8 hours in WT, UQCRC2 KO, and COX4 KO 143B cells expressing or not AOX and treated with DMSO or 500 nM antimycin (mean+/−SEM, N=3 per condition). D. DHODH activity as measured by stable isotope tracing with 10 mM ¹³C₄-aspartate, which generates ¹³C₃-UTP if DHODH is active. Tracing was for 8 hours in WT, SDHB KO, and KO 143B cells with the SDHB cDNA expressed and treated with DMSO or 500 nM antimycin (mean+/−SEM, N=3 per condition). E. First and last images from a live cell imaging video of WT and SDHB KO 143B cells treated with DMSO or 100 nM antimycin. F. Quantification of the mitochondrial membrane potential using live cell imaging of WT and SDHB KO 143B cells treated with 100 nM antimycin, which was added at the timepoint indicated by the arrow. G. Mitochondrial membrane potential of WT, SDHB KO, and SDHB KO cells expressing the SDHB cDNA treated with either DMSO or 500 nM antimycin for 1 hour (mean+/−SEM, N=3 per condition). H. Schematic depicting the hypothesis that expression of AOX will rescue complex I and DHODH activities in SDH KO cells treated with antimycin. I. Mitochondrial membrane potential in WT, SDHB KO, and SDHB KO 143B cells expressing AOX and treated with DMSO, 500 nM antimycin for 1 hour (mean+/−SEM, N=3 per condition). J. DHODH activity as measured via stable isotope tracing with 10 mM ¹³C₄-aspartate, which generates ¹³C₃-UTP if DHODH is active. Tracing was performed for 8 hours in WT, SDHB KO, and SDHB KO 143B cells expressing AOX and treated with DMSO or 500 nM antimycin (mean+/−SEM, N=3 per condition). For all experiments * indicates P<0.05. P values were calculated using an unpaired parametric t test.

FIG. 4 shows fumarate reduction supports mitochondrial functions in tissues capable of net-reversal of the SDH reaction. A. Depiction of the workflow for the in vivo ¹³C₅¹⁵N₂-glutamine stable isotope tracing experiment to measure the net-directionality of the SDH complex in tissues. B. In vivo stable isotope tracing of ¹³C₅¹⁵N₂-glutamine in indicated tissues. Mice were sacrificed 20 minutes post retroorbital and intraperitoneal injections. Succinate oxidation was calculated by the ratio of pmoles fumarate M+4:pmoles succinate M+4. Fumarate reduction was calculated by the ratio of pmoles succinate M+3:pmoles fumarate M+3 (mean+/−SEM, N=4 per condition). C. Tissue autonomous succinate oxidation or fumarate reduction as determined with ex vivo 2 mM ¹³C₅¹⁵N₂-glutamine stable isotope tracing for 24 hours in indicated tissues kept in a tissue culture incubator at 21% O₂ or a hypoxia incubator (1% O₂). Succinate oxidation and fumarate reduction were calculated as described in (B) (mean+/−SEM, N=4 per condition). D. In vivo ¹³C₅¹⁵N₂-glutamine tracing in female mice 12 weeks old via intraperitoneal and intramuscular injections. Mice were either rested, exercised for 30 minutes, or until exhaustion for approximately 1.5 hours, and then injected with ¹³C₅¹⁵N₂-glutamine. Post injection the rested mice were sacrificed 15 minutes later with no exercise, and the exercised mice continued to run on the treadmill for 15 minutes before being sacrificed. Tissues were harvested for metabolite isolation and mass spectrometry. Absolute quantification was performed to calculate the concentration of succinate M+3, succinate M+4, fumarate M+3, and fumarate M+4 in pmoles per μg tissue protein. The reported ratio representing fumarate reduction was calculated by the pmoles succinate M+3 per μg tissue protein:the pmoles fumarate M+3 per μg tissue protein. The reported ratio representing succinate oxidation was calculated by the pmoles fumarate M+4 per μg tissue protein:the pmoles succinate M+4 per μg tissue protein. Data represent the mean+/−SEM, N=5 mice per timepoint. For all experiments * indicates P<0.05. P values were calculated using an unpaired parametric t test. E. Ex vivo 3 mM ¹³C₄-aspartate stable isotope tracing for 16 hours in indicated tissues kept in an incubator at 21% or 1% O₂, or treated with 2 μM antimycin. Orotate M+4 levels reflect DHODH activity (mean+/−SEM, N=4 per condition). F. Model in which net-reversal of SDH supports certain mitochondrial functions in tissues under conditions that reduce electron transfer to O₂. For all experiments * indicates P<0.05. P values were calculated using an unpaired parametric t test.

FIG. 5 shows rhodoquinone detected in kidney, liver, and brain.

FIG. 6 shows rhodoquinone is detected at high levels in many tissues.

FIG. 7 shows expression of RquA allows for the generation of rhodoquinone in mammalian cells

FIG. 8 shows expression of RquA facilitates ubiquitous fumarate reduction and reduces oxygen consumption rate in mammalian cells.

FIG. 9 shows rhodoquinone depleted from kidney over time when cultured ex vivo.

FIG. 10 shows loss of rhodoquinone in the kidney coincides with a loss of fumarate reduction.

FIG. 11 shows rhodoquinones are detected in the stool and cecum.

FIG. 12 shows upstream intermediates that may be secreted from the microbiome.

FIG. 13 shows rhodoquinone and ubiquinone amounts in the kidney of wild-type and germ-free mice.

FIG. 14 shows RquA expression is doxycycline-inducible.

FIG. 15 shows rhodoquinone promotes the use of fumarate as a terminal electron acceptor in mammalian cells.

FIG. 16 shows rhodoquinone renders mammalian cells resistant to hypoxia.

FIG. 17 shows a western blot analysis if different protein levels in kidney derived cancer cell line that is cultured in hypoxia for 5 days. The results show that rhodoquinone protects against oxidative and bioenergetic stresses caused by hypoxia exposure. This includes NRF2 accumulation, which is a sensor of oxidative stress, and phosphorylation on PDH, which is a marker of AMPK activation. AMPK senses AMP, and is hyperactive when cells have “low energy”

FIG. 18 shows RquA supports complex 1 and DHODH activities. Proliferation of WT and RquA cells in media that forces cells to use maximal complex 1 activity (− pyruvate) and maximal DHODH activity (− uridine). Complex 1 and DHODH are enzymes in the ETC that require an electron carrier (classically UQ) to catalyze their function. The results show that RQ can carry electrons in the ETC and support complex 1 and DHODH activities. Rotenone was used as a control and inhibits complex 1 activity. Brequinar was used as a control and inhibits DHODH activity.

FIG. 19 shows an LCMS assay to measure mitochondrial ROS production in WT and RquA cells in normoxia and hypoxia. 2-OH-MitoE2+ is the oxidation product that happens when superoxide reacts with mitosox. 2-OH-MitoE2+ increases in WT cells exposed to hypoxia for 5 days. This increase is significantly blunted when RquA is expressed because rhodoquinone directs electrons onto complex II instead of complex III, which generates the majority of superoxide in cells exposed to hypoxia.

FIG. 20 shows that RquA blocks the increase in glucose consumption and lactate secretion caused by hypoxia exposure. Hypoxia normally increases glucose consumption and lactate secretion in cells because of disruption in electron flow in the ETC. When cells express RquA and direct electrons onto fumarate as an electron acceptor, this increased glucose consumption and lactate secretion in blunted because electron flow is not disrupted in the ETC upon hypoxia exposure.

FIG. 21 shows a schematic detailing how the ETC relates to glucose consumption and lactate secretion.

FIG. 22 shows a cell free assay on purified and permeablized mitochondria derived from WT and RquA-expressing cells. The results demonstrate that RQ is sufficient to drive fumarate reduction in mammalian mitochondria. This assay measures the rate of fumarate reduction in purified and permeablized mitochondria. Succinate production over time is monitored after challenging the mitochondria with fumarate and NADH, enabling them to perform fumarate reduction. RquA, in the absence of the complex III inhibitor antimycin, increases fumarate reduction compared to WT (UQ-containing) mitochondria.

FIG. 23 shows the results of ¹³C₅¹⁵N₂-glutamine tracing in WT and RquA-expressing cells to monitor the forward and reverse activities of the SDH complex.

FIG. 24 shows RquA decreases fatty acid oxidation in mammalian cells. RQ drives lower fatty acid oxidation in cells. Radioactivity assay to monitor 14C-palmitate degradation in WT (UQ-containing) and RquA (RQ-containing) 143B cells.

### DETAILED DESCRIPTION OF CERTAIN EMBODIMENTS OF THE INVENTION

### Methods of Treatment and Uses

The present disclosure provides methods of treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof, the method comprising administering to the subject a therapeutically effective amount of a compound of Formula (I):

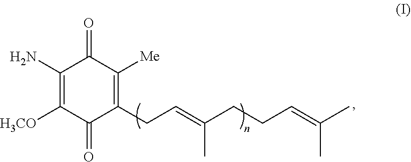

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof.

Also provided herein are methods of treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof, the method comprising:
administering to the subject a therapeutically effective amount of a compound of Formula (I):

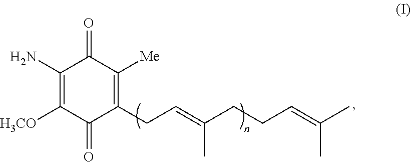

wherein n=1, 2, 3, 4, 5, 6, 7, 8, 9, 10, 11, or 12; or pharmaceutically acceptable salt, solvate, hydrate, polymorph, co-crystal, tautomer, stereoisomer, isotopically labeled derivative, or prodrug thereof; and
wherein the subject has previously been administered a statin.

Without wishing to be bound by any particular theory, statin treatment may lead to depletion of rhodoquinone in the subject, which may cause disease associated with depressed rhodoquinone levels (e.g., a proliferative disease, inflammatory disease, neuromuscular disorder, metabolic disorder, or neurodegenerative disorder).

These patients are also likely depleted of Rhodoquinone-10, and will likely need to also be supplementing their diets with this secondary electron carrier.

Also provided herein are methods of treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof, the method comprising:
administering to a subject in need thereof an agent or therapy that increases the level of a compound of Formula (I):

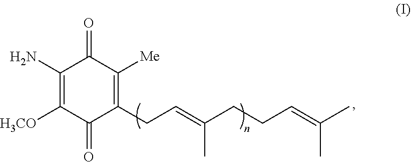

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; in the subject.

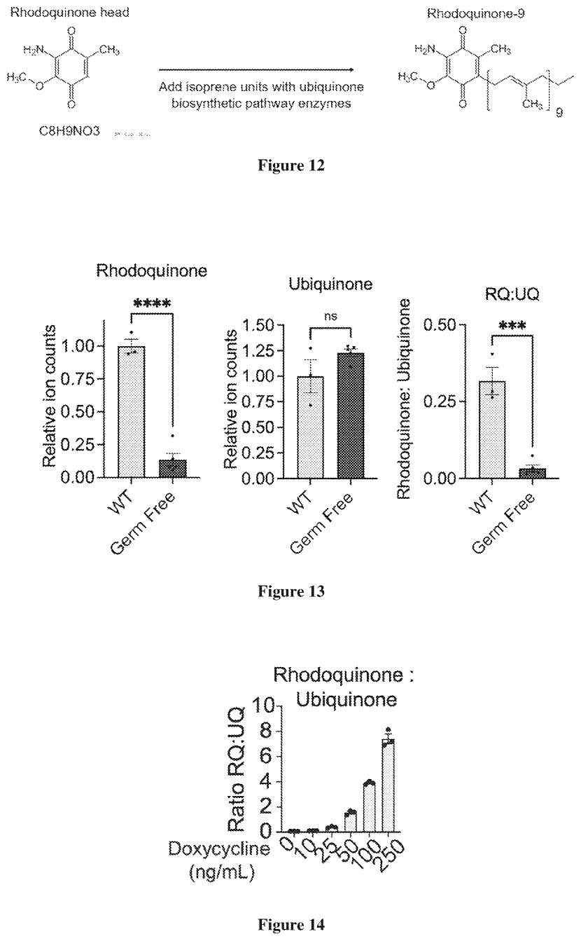

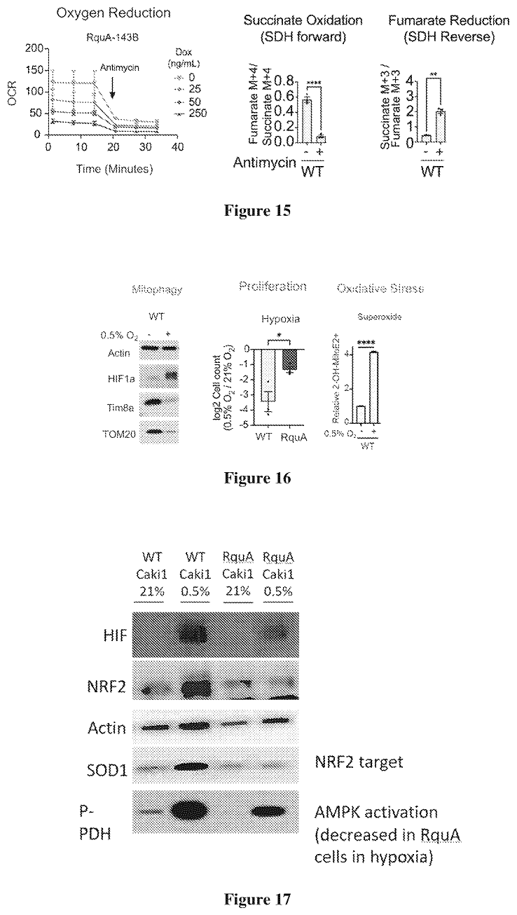

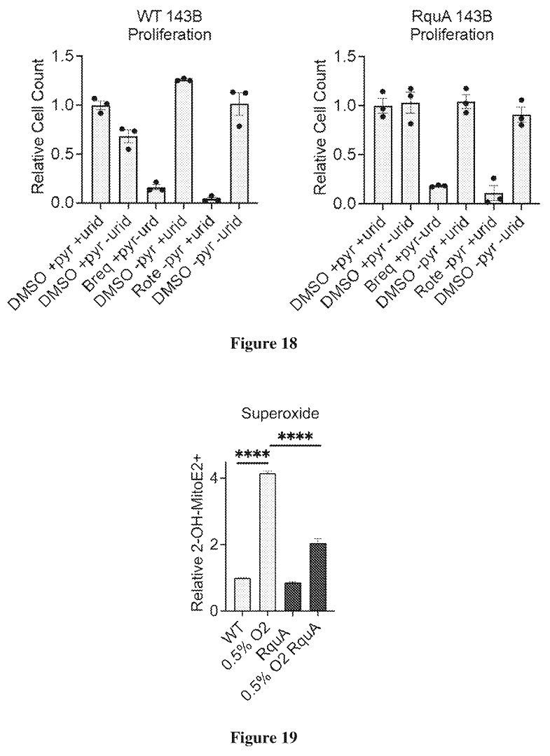

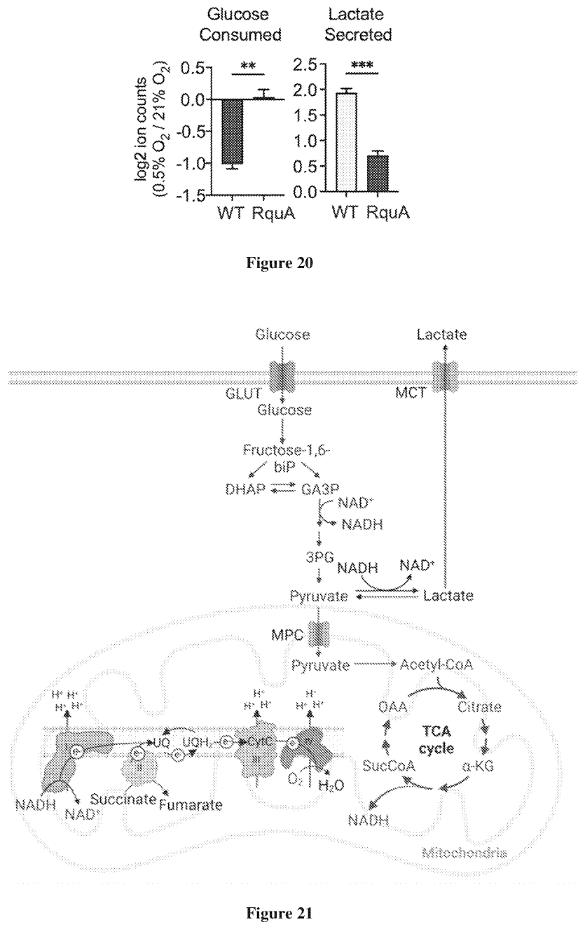

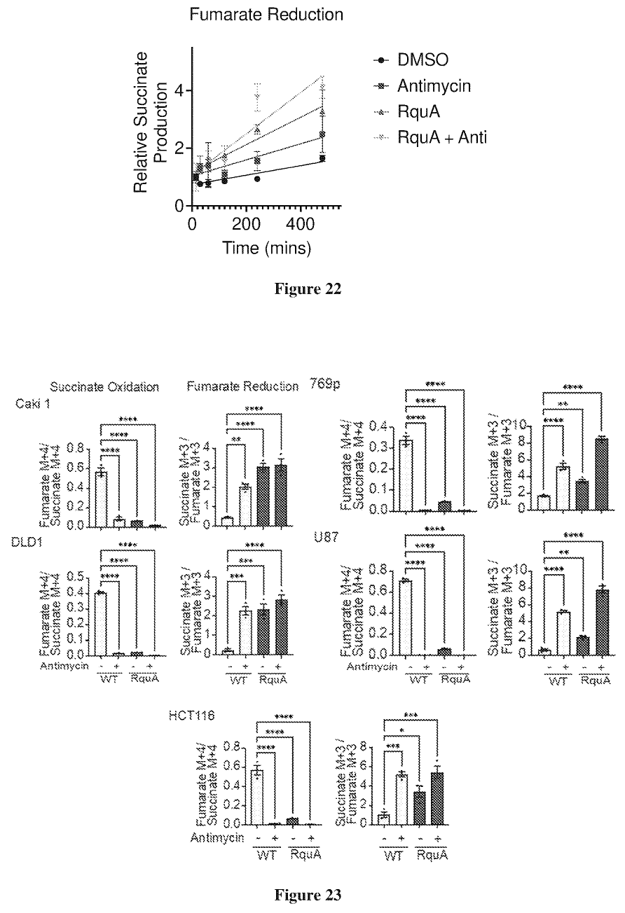

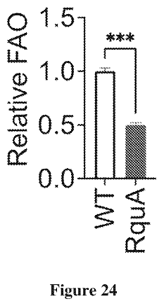

In certain embodiments, the agent is a compound of Formula (I) or Formula (II) or a pharmaceutically acceptable salt, solvate, hydrate, polymorph, co-crystal, tautomer, stereoisomer, isotopically labeled derivative, or prodrug thereof as described herein. In certain embodiments, the agent is a plasmid encoding a protein (e.g., an enzyme enabling in vivo conversion of ubiquinone to rhodoquinone (e.g., RquA)). In certain embodiments, the agent is a viral vector (e.g., a viral vector encoding a protein (e.g., an enzyme enabling in vivo conversion of ubiquinone to rhodoquinone (e.g., RquA)). Could also administer RNA encoding the protein. In certain embodiments, the therapy is a gene therapy. In certain embodiments, the therapy activates synthesis of rhodoquinone or rhodoquinone intermediates (e.g., as shown in FIG. 12). In certain embodiments, the therapy is a microbiome therapy. In certain embodiments, the microbiome therapy is bacterial supplementation into the microbiome.

Another aspect of the current disclosure relates to compounds of Formula (I):

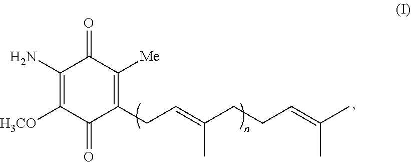

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof.

Also, provided herein is a compound of Formula (I):

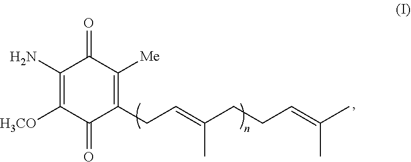

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in the manufacture of a medicament for the treatment of a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof.

Also, provided herein is the use of a compound of Formula (I):

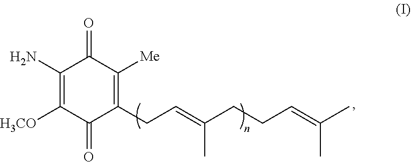

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for treating a disease (e.g., a proliferative disease, inflammatory disease, metabolic disorder, neuromuscular disorder, neurodegenerative disorder, hypoxia, ischemia, oxidative stress, or a mitochondrial DNA related disorder)) in a subject in need thereof.

Formula (I) contains n instance(s) of the isoprene unit. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10. In certain embodiments, n is 11. In certain embodiments, n is 12. In certain embodiments, n is 6, 7, 8, 9, 10, or 11. In certain embodiments, n is 8 or 9.

Another aspect of the disclosure relates to methods of treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof, the method comprising administering to the subject a therapeutically effective amount of a compound of Formula (II):

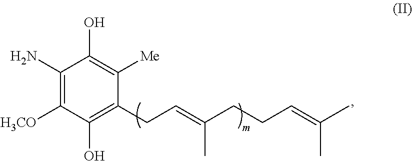

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof.

Also, provided herein are methods of treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof, the method comprising:
administering to the subject a therapeutically effective amount of a compound of Formula (II):

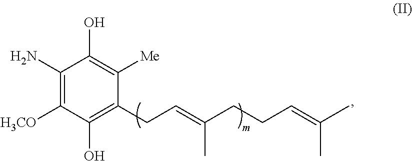

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof;
wherein the subject has previously been administered a statin.

Also, provided herein are methods of treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof, the method comprising:
administering to a subject in need thereof an agent or therapy that increases the level of a compound of Formula (II):

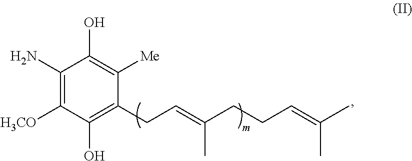

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; in the subject.

In certain embodiments, the agent is a compound of Formula (I) or Formula (II) or a pharmaceutically acceptable salt, solvate, hydrate, polymorph, co-crystal, tautomer, stereoisomer, isotopically labeled derivative, or prodrug thereof as described herein. In certain embodiments, the agent is a plasmid encoding an enzyme (e.g., Rqua). In certain embodiments, the therapy is a gene therapy. In certain embodiments, the gene therapy is an adeno-associated virus (AAV) vector. In certain embodiments, the therapy activates synthesis of rhodoquinone or rhodoquinone intermediates (e.g., as shown in FIG. 12). In certain embodiments, the therapy is a microbiome therapy. In certain embodiments, the microbiome therapy is bacterial supplementation into the microbiome.

Also provided herein is a compound of Formula (II):

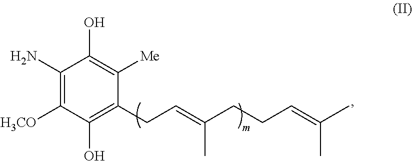

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof.

Also provided herein is a compound of Formula (II):

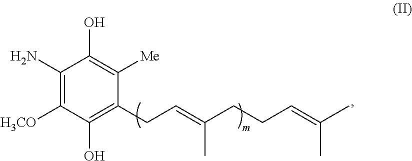

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in the manufacture of a medicament for the treatment of a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof.

Also provided herein is the use of a compound of Formula (II):

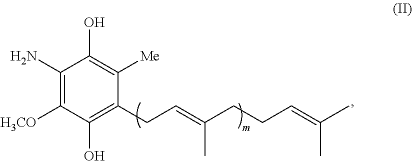

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for treating a disease (e.g., a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder)) in a subject in need thereof.

Formula (II) contains m instance of the isoprene unit. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 6, 7, 8, 9, 10, or 11. In certain embodiments, m is 8 or 9.

The present disclosure also provides a compound of Formula (I) or (II), or a pharmaceutically acceptable salt, solvate, hydrate, polymorph, co-crystal, tautomer, stereoisomer, isotopically labeled derivative, or prodrug thereof, which may be optionally administered in combination with an additional pharmaceutical agent, for example a vitamin, an antioxidant, an anti-inflammatory, an anti-cancer agent, an anti-obesity agent, a probiotic, an antibiotic, a statin, or a plasmid (e.g., a plasmid encoding a protein (e.g., an enzyme enabling in vivo conversion of ubiquinone to rhodoquinone (e.g., RquA))).

In certain embodiments, the disease is a proliferative disease, inflammatory disease, metabolic disorder, neuromuscular disorder, neurodegenerative disorder, hypoxia, ischemia, oxidative stress, or a mitochondrial DNA related disorder. In some embodiments a mitochondrial DNA disorder is characterized by one or more mutations in mitochondrial DNA. In certain embodiments, the disease is a proliferative disease. In certain embodiments, the disease is cancer. In certain embodiments, the cancer is associated with one or more mitochondrial mutations. In certain embodiments, the cancer is associated with one or more mitochondrial complex II mutations. In certain embodiments, the disease is a metabolic disorder. In certain embodiments, the disease is obesity.

Without wishing to be bound by any particular theory, rhodoquinone and/or rhodoquinol may stimulate weight loss by forcing cells to burn fuels like glucose and fatty acids more efficiently because rhodoquinone and/or rhodoquinol requires more fuel to be burned to generate energy in a subject.

In certain embodiments, the disease is diabetes. In certain embodiments, the disease is an inflammatory disorder. In certain embodiments, the disease is a neuromuscular disorder. In certain embodiments, the disease is a neurodegenerative disorder. In certain embodiments, the disease is hypoxia. In certain embodiments, the hypoxia is caused by obesity. In certain embodiments, the hypoxia is caused by lower atmospheric oxygen content (e.g., at higher altitudes). In certain embodiments, the disease is mitophagy. In certain embodiments, the disease is ischemia. In certain embodiments, the disease is ischemia-reperfusion injury. In certain embodiments, the ischemia is ischemia of the heart. In certain embodiments, the ischemia is ischemia of the kidney. In certain embodiments, the ischemia is ischemia of the liver. In certain embodiments, the ischemia is caused by coronary artery disease. In certain embodiments, the ischemia is ischemia of the central nervous system. In certain embodiments, the ischemia is ischemia of the brain. In certain embodiments, the ischemia is caused by cardiomyopathy. In certain embodiments, the ischemia is caused by alcoholic cardiomyopathy. In certain embodiments, the ischemia is caused by angioplasty. In certain embodiments, the ischemia is caused by stenting. In certain embodiments, the ischemia is caused by heart surgery such as bypass surgery or heart repair surgery (“open-heart surgery”). In certain embodiments, the ischemia is caused by organ transplantation. In certain embodiments, the ischemia is caused by prolonged weight pressure on tissues (pressure ulcers or bedsores). In certain embodiments, the ischemia is caused by ischemia-reperfusion injury which can cause damage to transplanted organs or tissue. In certain embodiments, the disease is oxidative stress. In certain embodiments, the oxidative stress is caused by elevated intracellular levels of reactive oxygen species (ROS). In certain embodiments, the oxidative stress is acetaminophen-induced. In certain embodiments, the disease is a mitochondrial DNA related disorder. In certain embodiments, the disease is CoQ10 deficiency. In certain embodiments, the disease is mitochondrial complex 1 deficiency. In certain embodiments, the disease is mitochondrial complex 2 deficiency. In certain embodiments, the disease is mitochondrial complex 3 deficiency. In certain embodiments, the disease is mitochondrial complex 4 deficiency.

In certain embodiments, provided herein are methods of inhibiting the Nrf2 pathway in a subject in need thereof, comprising administering to the subject a therapeutically effective amount of a compound of Formula (I):

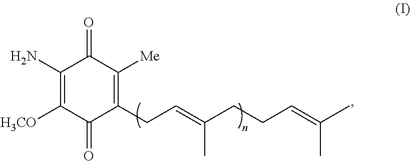

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof.

In certain embodiments, provided herein are methods of inhibiting the Nrf2 pathway in a subject in need thereof, administering to a subject in need thereof a compound of Formula (II):

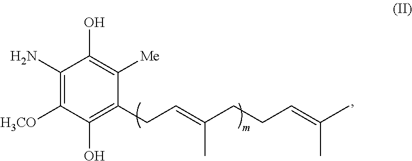

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; in the subject.

In certain embodiments, provided herein are methods of decreasing AMPK activation in a subject in need thereof, comprising administering to the subject a therapeutically effective amount of a compound of Formula (I):

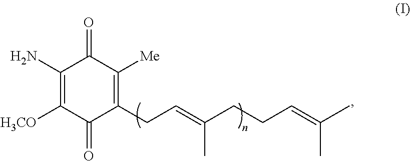

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof.

In certain embodiments, provided herein are methods of decreasing AMPK activation in a subject in need thereof, comprising administering to a subject in need thereof a compound of Formula (II):

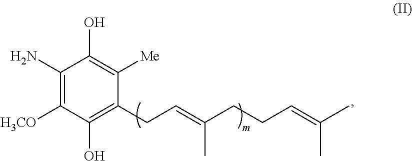

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; in the subject.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal, such as a transgenic mouse or transgenic pig.

In certain embodiments, the biological sample being contacted with the compound of Formula (I) or Formula (II), or pharmaceutical composition thereof, is breast tissue, bone marrow, lymph node, lymph tissue, spleen, or blood. In certain embodiments, the biological sample being contacted with the compound or pharmaceutical composition thereof is a tumor or cancerous tissue. In certain embodiments, the biological sample being contacted with the compound or pharmaceutical composition thereof is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from the biological sample.

In certain embodiments, the cell or tissue being contacted with the compound or pharmaceutical composition thereof is present in vitro. In certain embodiments, the cell or tissue being contacted with the compound or pharmaceutical composition thereof is present in vivo. In certain embodiments, the cell or tissue being contacted with the compound or pharmaceutical composition thereof is present ex vivo. In certain embodiments, the cell or tissue being contacted is a hypoxic cell or tissue. In certain embodiments, the cell or tissue being contacted is an ischemic cell or tissue. In certain embodiments, the cell or tissue being contacted with the compound or pharmaceutical composition thereof is a malignant cell (e.g., malignant blood cell). In certain embodiments, the cell being contacted with the compound or pharmaceutical composition thereof is a malignant hematopoietic stem cell (e.g., malignant myeloid cell or malignant lymphoid cell). In certain embodiments, the cell being contacted with the compound or pharmaceutical composition thereof is a malignant lymphocyte (e.g., malignant T-cell or malignant B-cell). In certain embodiments, the cell being contacted with the compound or pharmaceutical composition thereof is a malignant white blood cell. In certain embodiments, the cell being contacted with the compound or pharmaceutical composition thereof is a malignant neutrophil, malignant macrophage, or malignant plasma cell. In certain embodiments, the cell being contacted with the compound or pharmaceutical composition thereof is a carcinoma cell. In certain embodiments, the cell being contacted with the compound or pharmaceutical composition thereof is a breast carcinoma cell. In certain embodiments, the cell being contacted with the compound or pharmaceutical composition thereof is a sarcoma cell. In certain embodiments, the cell being contacted with the compound or pharmaceutical composition thereof is a sarcoma cell from breast tissue. In certain embodiments, the biological sample is from tissue or cells with cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma). In certain embodiments, the biological sample is from tissue or cells with an inflammatory disease or autoimmune disease. In certain embodiments, the biological sample is from tissue or cells with cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma), an inflammatory disease, or an autoimmune disease.

All types of biological samples described herein or known in the art are contemplated as being within the scope of the invention. In certain embodiments, the disease (e.g., a proliferative disease, inflammatory disease, metabolic disorder, neuromuscular disorder, neurodegenerative disorder, hypoxia, ischemia, oxidative stress, or a mitochondrial DNA related disorder) to be treated or prevented using the compounds described herein is cancer. All types of cancers disclosed herein or known in the art are contemplated as being within the scope of the invention. In certain embodiments, the proliferative disease is a hematological malignancy. In certain embodiments, the proliferative disease is a blood cancer. In certain embodiments, the proliferative disease is a hematological malignancy. In certain embodiments, the proliferative disease is leukemia. In certain embodiments, the proliferative disease is chronic lymphocytic leukemia (CLL). In certain embodiments, the proliferative disease is acute lymphoblastic leukemia (ALL). In certain embodiments, the proliferative disease is T-cell acute lymphoblastic leukemia (T-ALL). In certain embodiments, the proliferative disease is chronic myelogenous leukemia (CML). In certain embodiments, the proliferative disease is acute myeloid leukemia (AML). In certain embodiments, the proliferative disease is acute monocytic leukemia (AMoL). In certain embodiments, the proliferative disease is Waldenström's macroglobulinemia. In certain embodiments, the proliferative disease is Waldenström's macroglobulinemia associated with the MYD88 L265P somatic mutation. In certain embodiments, the proliferative disease is myelodysplastic syndrome (MDS). In certain embodiments, the proliferative disease is a carcinoma. In certain embodiments, the proliferative disease is lymphoma. In certain embodiments, the proliferative disease is T-cell lymphoma. In some embodiments, the proliferative disease is Burkitt's lymphoma. In certain embodiments, the proliferative disease is a Hodgkin's lymphoma. In certain embodiments, the proliferative disease is a non-Hodgkin's lymphoma. In certain embodiments, the proliferative disease is multiple myeloma. In certain embodiments, the proliferative disease is melanoma. In certain embodiments, the proliferative disease is colorectal cancer. In certain embodiments, the proliferative disease is colon cancer. In certain embodiments, the proliferative disease is breast cancer. In certain embodiments, the proliferative disease is recurring breast cancer. In certain embodiments, the proliferative disease is mutant breast cancer. In certain embodiments, the proliferative disease is HER2+ breast cancer. In certain embodiments, the proliferative disease is HER2− breast cancer. In certain embodiments, the proliferative disease is triple-negative breast cancer (TNBC). In certain embodiments, the proliferative disease is a bone cancer. In certain embodiments, the proliferative disease is osteosarcoma. In certain embodiments, the proliferative disease is Ewing's sarcoma. In some embodiments, the proliferative disease is a brain cancer. In some embodiments, the proliferative disease is neuroblastoma. In some embodiments, the proliferative disease is a lung cancer. In some embodiments, the proliferative disease is small cell lung cancer (SCLC). In some embodiments, the proliferative disease is non-small cell lung cancer (NSCLC). In certain embodiments, the lung cancer is mesothelioma. In certain embodiments, the cancer is a thyroid cancer. In certain embodiments, the cancer is a sarcoma. In certain embodiments, the sarcoma is Kaposi's sarcoma. In certain embodiments, the cancer is fallopian tube cancer. In certain embodiments, the cancer is a carcinoma. In certain embodiments, the carcinoma is fallopian tube carcinoma. In some embodiments, the proliferative disease is liver cancer. In some embodiments, the proliferative disease is prostate cancer. In some embodiments, the proliferative disease is pancreatic cancer. In some embodiments, the proliferative disease is gastric cancer. In some embodiments, the proliferative disease is ovarian cancer. In some embodiments, the proliferative disease is ovarian cancer. In some embodiments, the cancer is skin cancer. In some embodiments, the cancer is esophageal cancer. In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention. In certain embodiments, the cancer is a sarcoma, lung cancer, thyroid cancer, breast cancer, liver cancer, pancreatic cancer, gastric cancer, ovarian cancer, colon cancer, colorectal cancer, skin cancer, esophageal cancer; or a carcinoma.

In certain embodiments, the disease is an inflammatory disease. In certain embodiments, the inflammatory disease to be treated or prevented using the compounds described herein is fibrosis (e.g., idiopathic pulmonary fibrosis, liver cirrhosis, cystic fibrosis, systemic sclerosis, progressive kidney disease, or cardiovascular fibrosis).

In certain embodiments, the autoimmune disease to be treated or prevented using the compounds described herein is sclerosis (e.g., systemic sclerosis (scleroderma) or multiple sclerosis).

In certain embodiments, the methods described herein include administering to a subject or contacting a biological sample with an effective amount of a compound described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the methods described herein include administering to a subject or contacting a biological sample with an effective amount of a compound described herein, or a pharmaceutically acceptable salt thereof, or a pharmaceutical composition thereof. In certain embodiments, the compound is contacted with a biological sample. In certain embodiments, the compound is administered to a subject. In certain embodiments, the compound is administered in combination with one or more additional pharmaceutical agents described herein. The additional pharmaceutical agent may be an anti-proliferative agent. In certain embodiments, the additional pharmaceutical agent is an anti-cancer agent. In certain embodiments, the additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-obesity agents, anti-diabetic agents, anti-allergic agents, contraceptive agents, pain-relieving agents, probiotic, antibiotic, statin, or plasmid (e.g., a plasmid encoding RquA), and any combination thereof. In certain embodiments, the additional pharmaceutical agent is an anti-obesity agent. In certain embodiments, the additional pharmaceutical agent is a probiotic. In certain embodiments, the additional pharmaceutical agent is an antibiotic. In certain embodiments, the additional pharmaceutical agent is a statin. In certain embodiments, the additional pharmaceutical agent is plasmid (e.g., a plasmid encoding RquA).

In some embodiments, the additional pharmaceutical agent is an anti-obesity agent. In some embodiments, the additional pharmaceutical agent is a probiotic. In some embodiments, the additional pharmaceutical agent is an antibiotic. In some embodiments, the additional pharmaceutical agent is a statin. In some embodiments, the additional pharmaceutical agent is a plasmid (e.g., a plasmid encoding RquA). In certain embodiments, a pharmaceutical composition described herein further comprises a combination of the additional pharmaceutical agents described herein.

### Pharmaceutical Compositions, Kits, and Administration

The present disclosure also provides pharmaceutical compositions comprising a compound of Formula (I) or Formula (II) as described herein and optionally a pharmaceutically acceptable excipient. In certain embodiments, the composition comprises a therapeutically effective amount of a compound of Formula (I):

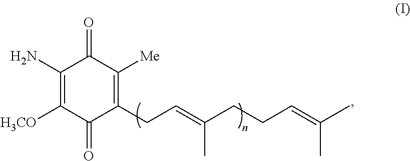

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, and an additional pharmaceutical agent.

In certain embodiments, the composition comprises a therapeutically effective amount of a compound of Formula (II):

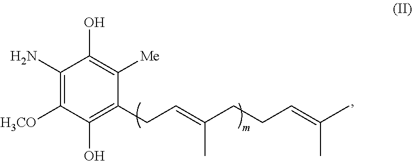

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, and an additional pharmaceutical agent.

In certain embodiments, a compound described herein is a compound of Formula (I) or (II), or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount.

In certain embodiments, the cell being contacted with a compound or pharmaceutical composition thereof described herein is in vitro. In certain embodiments, the cell being contacted with a compound or pharmaceutical composition thereof described herein is in vivo.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragées, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample (e.g., tissue, cell), any two doses of the multiple doses include different or substantially the same amounts of a compound described herein. In certain embodiments, when multiple doses are administered to a subject or applied to a biological sample (e.g., tissue, cell), the frequency of administering the multiple doses to the subject or applying the multiple doses to the biological sample (e.g., tissue, cell) is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample (e.g., tissue, cell) is one dose per day. In certain embodiments, the frequency of administering the multiple doses to the subject or applying the multiple doses to the biological sample (e.g., tissue, cell) is two doses per day. In certain embodiments, the frequency of administering the multiple doses to the subject or applying the multiple doses to the biological sample (e.g., tissue, cell) is three doses per day. In certain embodiments, when multiple doses are administered to a subject or applied to a biological sample (e.g., tissue, cell), the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The compound or pharmaceutical composition thereof can be administered concurrently with, prior to, or subsequent to one or more additional pharmaceutical agents (e.g., a statin), which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, inflammatory disease, autoimmune disease). Each additional pharmaceutical agent may be administered at a dose and/or on a time schedule determined for that pharmaceutical agent. The additional pharmaceutical agents may also be administered together with each other and/or with the compound or pharmaceutical composition thereof described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-obesity agents, anti-diabetic agents, anti-allergic agents, contraceptive agents, pain-relieving agents, probiotic, antibiotic, statin, or plasmid, and any combination thereof. In certain embodiments, the additional pharmaceutical agent is an anti-obesity agent. In certain embodiments, the additional pharmaceutical agent is a probiotic. In certain embodiments, the additional pharmaceutical agent is an antibiotic. In certain embodiments, the additional pharmaceutical agent is a statin. In certain embodiments, the additional pharmaceutical agent is plasmid (e.g., a plasmid encoding RquA).

Also encompassed by the disclosure are kits (e.g., pharmaceutical packs). In certain embodiments, a kit as described herein comprises:
a compound of Formula (I):

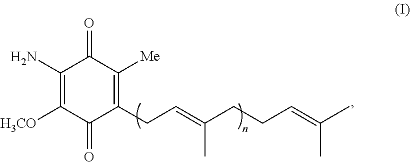

wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; and
instructions for using the compound, or a pharmaceutically acceptable salt, solvate, hydrate, polymorph, co-crystal, tautomer, stereoisomer, isotopically labeled derivative, or prodrug thereof.

In certain embodiments, a kit as described herein comprises:
a compound of Formula (II):

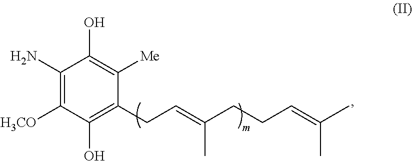

wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; and
instructions for using the compound, or pharmaceutically acceptable salt, solvate, hydrate, polymorph, co-crystal, tautomer, stereoisomer, isotopically labeled derivative, or prodrug thereof.

The kits provided may comprise a pharmaceutical composition or compound of Formula (I) or Formula (II) as described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., a proliferative disease, inflammatory disease, metabolic disorder, neuromuscular disorder, neurodegenerative disorder, hypoxia, ischemia, oxidative stress, or a mitochondrial DNA related disorder) in a subject in need thereof. In certain embodiments, the kits are useful for preventing a disease (e.g., a proliferative disease, inflammatory disease, metabolic disorder, neuromuscular disorder, neurodegenerative disorder, hypoxia, ischemia, oxidative stress, or a mitochondrial DNA related disorder) in a subject in need thereof.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., a proliferative disease, inflammatory disease, metabolic disorder, neuromuscular disorder, neurodegenerative disorder, hypoxia, ischemia, oxidative stress, or a mitochondrial DNA related disorder) in a subject in need thereof. In certain embodiments, the kits and instructions provide for preventing a disease (e.g., a proliferative disease, inflammatory disease, metabolic disorder, neuromuscular disorder, neurodegenerative disorder, hypoxia, ischemia, oxidative stress, or a mitochondrial DNA related disorder) in a subject in need thereof.

In certain embodiments, a kit described herein includes a first container comprising a compound or pharmaceutical composition described herein. In certain embodiments, a kit described herein is useful in treating and/or preventing a disease, such as a metabolic disorder (e.g., obesity, diabetes), a hypoxia related disease (e.g., a proliferative disease, inflammatory disease, neuromuscular disorder, neurodegenerative disorder, hypoxia, ischemia, oxidative stress, or a mitochondrial DNA related disorder), or a disease resulting from rhodoquinone depletion (e.g., a proliferative disease, inflammatory disease, neuromuscular disorder, metabolic disorder, or neurodegenerative disorder).

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease in a subject in need thereof, preventing a disease, such as a proliferative disease, inflammatory disease, metabolic disorder, neuromuscular disorder, neurodegenerative disorder, hypoxia, ischemia, oxidative stress, or a mitochondrial DNA related disorder. A kit described herein may include one or more additional pharmaceutical agents described herein as a separate composition.

### EXAMPLES

In order that the disclosure described herein may be more fully understood, the following examples are set forth. The biological examples described in this application are offered to illustrate the compounds, pharmaceutical compositions, and methods provided herein and are not to be construed in any way as limiting their scope.

### Example 1. In Mammalian Cells the Flow of Electrons into the ETC does not Require O2 Reduction

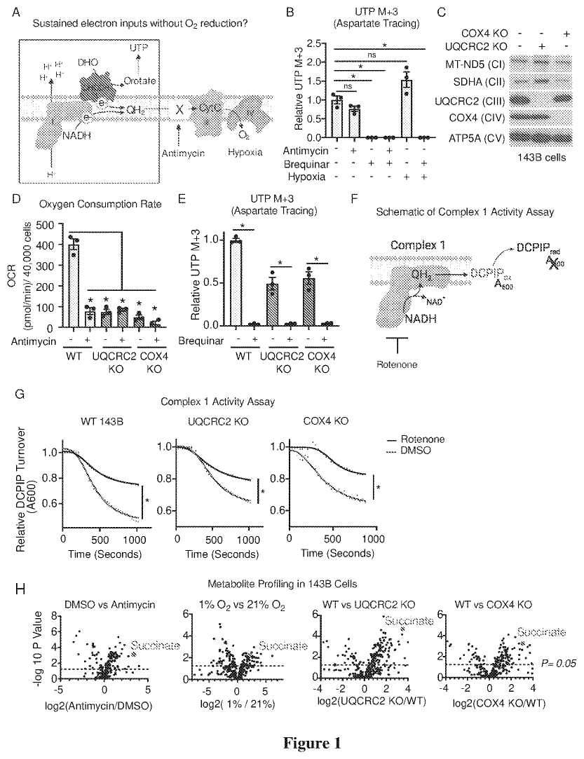

DHODH oxidizes dihydroorotate into orotate and deposits these electrons into the ETC (FIG. 1A). Since ETC inhibition reduces aspartate levels, using aspartate as the tracer ensures that its availability is not limiting for DHODH activity. Treatment of human 143B cells with antimycin, which inhibits complex III and thus prevents the transfer of electrons to O₂ by complex IV, did not decrease de novo UTP biosynthesis as followed by the generation of ¹³C₃-UTP (FIG. 1B). In contrast, in both vehicle and antimycin-treated cells, the DHODH inhibitor brequinar ablated ¹³C₃-UTP production (FIG. 1B). Similarly, under hypoxia (1% O₂), cells sustained pyrimidine biosynthesis in a DHODH-dependent manner (FIG. 1B). Thus, under conditions that reduce the transfer of electrons to O₂, DHODH can still deposit electrons into the ETC.

Because complex IV has affinity for O₂ and thus can have partial activity even under hypoxia, 143B cells were generated that are genetically incapable of reducing O₂ because they lack a key component of complex IV (COX4) or complex III (UQCRC2). Loss of these genes did not induce the expression of paralogues that might substitute for them, reduce the levels or assembly of the other ETC complexes, or strongly impact MT-DNA copy number (FIG. 1C). While in the UQCRC2 and COX4 knockout cells O₂ consumption was greatly reduced (FIG. 1D), de novo pyrimidine biosynthesis was less impaired. DHODH enzyme activity was unaffected by the loss of UQCRC2 or COX4 in cell lysates, and dropped ˜50% from wild-type levels in live cells as measured by ¹³C₄-aspartate incorporation into ¹³C₃-UTP (FIG. 1E). The DHODH inhibitor brequinar ablated ¹³C₃-UTP biosynthesis in UQCRC2 and COX4 knockout cells despite no effect on their O₂ consumption rate (FIG. 1E), indicating that DHODH maintains activity in these cells independent of their ability to reduce O₂ (FIG. 1E). Furthermore, supplementation of culture media with aspartate, an essential precursor in de novo pyrimidine biosynthesis, increased the proliferation of antimycin-treated cells, which was ablated by the DHODH inhibitor brequinar. Similarly, brequinar treatment reduced the proliferation of UQCRC2 and COX4 knockout cells. Thus, both pharmacological and genetic experiments reveal DHODH maintains electron input into the ETC even when O₂ cannot be used as a terminal electron acceptor, and that adaptive mechanisms must exist to sustain electron flow into the ETC in this context.

To determine if other electron inputs into the ETC are maintained when O₂ cannot be utilized as the terminal electron acceptor, complex I activity was examined because it deposits electrons into the ETC during the oxidation of NADH. Mitochondria were purified from wild-type, UQCRC2- and COX4-knockout 143B cells and measured complex I enzymatic activity (FIG. 1F). All mitochondria, including those genetically incapable of O₂ reduction, had significantly reduced complex I activity upon rotenone treatment, indicating that complex I enzymatic activity is intact (FIG. 1G). The activity of complex I was slightly lower in mitochondria lacking UQCRC2 or COX4 (FIG. 1G), consistent with previous findings that hypoxia reduces but does not ablate complex I activity in cells. Inhibition of complex I by piericidin did not affect O₂ consumption rate in UQCRC2 and COX4 knockout cells, but did reduce their proliferation, although to a lesser extent than in wild-type cells. Thus, as with DHODH, complex I can still deposit electrons into the ETC when O₂ cannot be reduced.

### Example 2. Inhibition of O₂ Reduction Stimulates a Net-Reversal of the SDH Complex, Enabling Fumarate Reduction Mammalian Cells

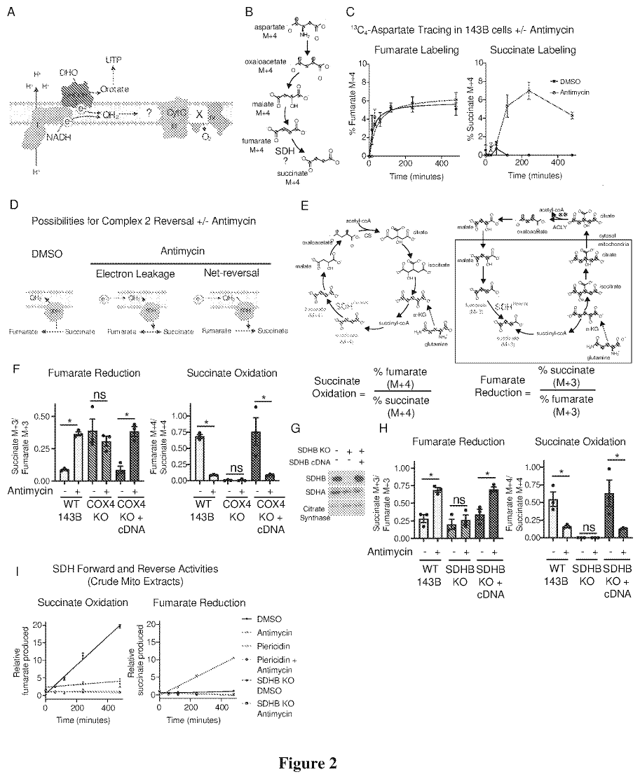

DHODH and complex I can deposit electrons into the ETC even when O₂ reduction is not possible (FIG. 2A). Upon exposure to hypoxia, electrons can be transferred to NAD+ through reversal of complex I activity. Combined inhibition of complex III with antimycin and complex I with piericidin had no effect on DHODH activity as measured by the incorporation of ¹³C₄-aspartate into ¹³C₃-UTP, indicating that an alternative electron removal pathway must sustain nucleotide biosynthesis in the absence of O₂ reduction. Under hypoxia, lower eukaryotes such as C. elegans, yeast, and parasitic helminths use fumarate as a terminal electron acceptor, generating succinate as a by-product. Succinate also accumulates in cancer cells exposed to hypoxia, ischemic hearts, and post-exercise muscle. In 143B cells an increase in succinate upon hypoxia exposure was found, antimycin treatment, and depletion of UQCRC2 or COX4 (FIG. 1H).

Stable isotope tracing studies demonstrate that the majority of the succinate pool in hypoxic cells derives from α-ketoglutarate via oxidative TCA cycle flux. However, numerous studies find that a significant fraction of the succinate pool comes from fumarate upon a block in O₂ reduction. This reaction is likely catalyzed by the succinate dehydrogenase (SDH) protein complex (complex II).

Fumarate reduction can be monitored in cells by the stable isotope tracing of either ¹³C₄-aspartate or ¹³C₅¹⁵N₂-glutamine. ¹³C₄-aspartate contributes to the fumarate pool via oxaloacetate and malate and so should lead to the production of ¹³C₄-succinate upon fumarate reduction (FIG. 2B). ¹³C₅¹⁵N₂-glutamine contributes to the fumarate pool through the reductive arm of the TCA cycle via glutamate, α-ketoglutarate, isocitrate, citrate, oxaloacetate, and malate. A key distinction between the two labeling approaches is that ¹³C₅¹⁵N₂-glutamine enriches the fumarate pool more upon antimycin treatment due to enhanced reductive carboxylation flux, whereas ¹³C₄-aspartate labels the fumarate pool to equivalent extents in vehicle and antimycin-treated cells (FIG. 2C). Therefore, with stable isotope tracing of ¹³C₄-aspartate, the level of ¹³C₄-succinate is a direct measure of fumarate reduction, whereas the stable isotope tracing of ¹³C₅¹⁵N₂-glutamine requires a ratiometric analysis of labeled succinate to labeled fumarate to normalize for differences in the extent of fumarate labeling.

Antimycin robustly stimulated the conversion of fumarate into succinate, as monitored by the production of ¹³C₄-succinate over time and the ratio of %¹³C₄-succinate:% ¹³C₄-fumarate when labeling was in the steady-state after 8 hours of incubation with ¹³C₄-aspartate (FIG. 2C).

To test if hypoxia triggers electron leakage onto fumarate or a net-reversal of the SDH complex, the ¹³C₅¹⁵N₂-glutamine tracer was utilized, which enables simultaneous quantification of the forward and reverse activities of SDH through the generation of unique isotopologues upon flux through the oxidative arm (α-ketoglutarate to succinate) compared to the reductive arm (α-ketoglutarate—via citrate—to fumarate) of the TCA cycle (FIG. 2E). In this assay, the forward (succinate oxidation) reaction was defined as the ratio of percent labeled ¹³C₄-fumarate to its precursor ¹³C₄-succinate (FIG. 2E) and the reverse (fumarate reduction) as that of percent labeled ¹³C₃-succinate to its precursor ¹³C₃-fumarate (FIG. 2E). This type of analysis can be performed when the labeling is in the steady-state, which was determined to be after 4-8 hours of incubation with ¹³C₅¹⁵N₂-glutamine (FIG. S2E-F). This ratiometric analysis of the succinate and fumarate isotopologues eliminates potential biases in the assay, such as higher ¹³C₃-fumarate labeling in antimycin-treated cells caused increased reductive carboxylation flux (FIG. S2E).

Inhibition of O₂ reduction by antimycin or hypoxia decreased succinate oxidation and increased fumarate reduction, which are the SDH forward and reverse activities, respectively (FIG. 2F). Because the ratio of isotopologues representing fumarate reduction exceeded those for succinate oxidation by approximately 4-fold, antimycin treatment and hypoxia exposure cause higher levels of fumarate reduction than succinate oxidation. Likewise, the UQCRC2 and COX4 knockout cells had approximately 6- and 8-fold higher levels of fumarate reduction than succinate oxidation, respectively, whereas the opposite was true in the control cells (FIG. 2F). Expression in the knockout cells of the UQCRC2 or COX4 cDNA rescued O₂ consumption and restored succinate oxidation and fumarate reduction reactions and their sensitivity to antimycin treatment to close to wild-type levels (FIG. 2F). In cells lacking either the SDHA or SDHB component of the SDH complex, the fumarate reduction and succinate oxidation reactions were not altered by antimycin treatment, indicating a critical role for the SDH complex in mediating these reactions. Expression in the knockout cells of the respective cDNAs restored the increase in fumarate reduction and decrease in succinate oxidation caused by antimycin treatment. The complex II inhibitor malonic acid almost completely ablated fumarate reduction in UQCRC2 and COX4 knockout cells and had no effect on SDHB knockout cells. Treatment of 143B cells with dimethylsuccinate slightly suppressed the increase in fumarate reduction caused by antimycin treatment, consistent with high succinate levels inhibiting SDH activity.

In the untreated wild-type, SDHA, and SDHB knockout cells, background levels of fumarate reduction were detected, likely caused by the non-enzymatic reduction of fumarate into succinate, consistent with its electrophilic nature. To test if electron leakage out of the ETC contributes to background fumarate reduction, 143B cells were treated with the mitochondrial-targeted antioxidant mitoTEMPO in the presence or absence of antimycin. MitoTEMPO caused a significant decrease in fumarate reduction in vehicle-treated cells, but did not affect fumarate reduction in antimycin-treated cells, suggesting that electron leakage may contribute to baseline levels of fumarate reduction.

Antimycin treatment also increased fumarate reduction in a panel of other human cancer cell lines (SW1353, U87, DLD1, and HCT116), in the mouse myoblast cell line C2C12, primary dermal fibroblasts, and Mink lung epithelial cells.

SDH activity was examined in permeabilized purified mitochondria. The rate of succinate oxidation was measured by monitoring fumarate production over time after initiating the reaction with succinate. The rate of fumarate reduction was measured in a separate assay by monitoring the rate of succinate production over time after initiating the reaction with fumarate and NADH. Consistent with the stable isotope tracing experiments in live cells, vehicle-treated mitochondria had a higher rate of succinate oxidation than fumarate reduction, whereas the opposite was the case in antimycin-treated mitochondria (FIG. 2I). Mitochondria lacking the intact SDH complex did not exhibit any succinate oxidation or fumarate reduction. Moreover, complex I inhibition by piericidin suppressed the fumarate reduction caused by antimycin treatment (FIG. 2I).

### Example 3. Ubiquinol Accumulation is Required for SDH Reversal In Vitro

Net-reversal of the mammalian SDH complex has been considered thermodynamically unfavorable because the standard reduction potential of ubiquinone is slightly greater than that of fumarate. Moreover, unlike lower eukaryotes, mammals do not possess a distinct electron carrier with a lower reduction potential that could facilitate fumarate reduction. Because the reduction potential of ubiquinone (UQ) and fumarate are very close to each other, approximately 10 mV apart). The yeast protein, alternative oxidase (AOX), which can oxidize UQH₂ to UQ in an antimycin-insensitive manner was tested to determine if UQH₂ accumulation is necessary to drive SDH in reverse.

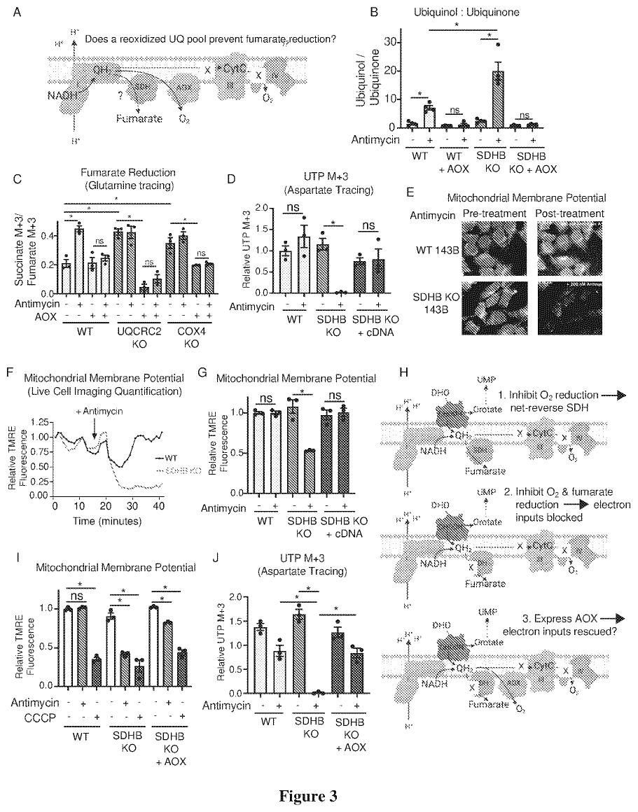

The AOX was targeted to the mitochondrial inner membrane, where, consistent with its role in maintaining an oxidized UQ pool, it blunted NADH and UQH₂ accumulation upon antimycin treatment (FIG. 3B). The levels of UQH₂ and the ratio of UQH₂:UQ were greater in antimycin-treated SDHB knockout cells than in antimycin-treated wild-type cells, consistent with the SDH complex playing a critical role in UQH₂ reoxidation when O₂ cannot be used as the terminal electron acceptor (FIG. 3B). Importantly, expression of AOX in SDHB knockout cells blunted the accumulation of UQH₂ upon antimycin treatment (FIG. 3B). Expression of AOX in UQCRC2 and COX4 knockout cells reduced the UQH₂: UQ ratio and the levels of UQH₂.

To determine if UQH₂ accumulation is required to reverse the SDH complex upon a block in O₂ reduction, a stable isotope tracing was used of both ¹³C₅¹⁵N₂-glutamine and ¹³C₄-aspartate to measure fumarate reduction in AOX-expressing wild-type cells as well as those lacking UQCRC2 or COX4 (FIG. 3C). Consistent with the idea that UQH₂ accumulation is required to initiate fumarate reduction upon inhibition of O₂ reduction, AOX expression fully suppressed the increase in fumarate reduction caused by antimycin treatment in wild-type cells (FIG. 3C). AOX expression in the UQCRC2 and COX4 knockout cells, which are genetically incapable of transferring electrons to O₂ and always operate the SDH complex in reverse, also suppressed fumarate reduction and almost completely restored succinate oxidation to wild-type levels (FIG. 3C).

The impact of AOX expression on SDH directionality was corroborated using permeabilized purified mitochondria from AOX-expressing 143B cells treated with vehicle or antimycin. As before (FIG. 2I), the SDH forward (succinate oxidation) and reverse (fumarate reduction) reactions were monitored over time by measuring the conversion of succinate to fumarate and fumarate to succinate, respectively. Consistent with the stable isotope tracing results in live cells, AOX expression prevented fumarate reduction upon antimycin treatment. Taken together, these data demonstrate that when O₂ reduction is blocked, UQH₂ accumulation is required for SDH reversal.

### Example 4. Fumarate Reduction Sustains Electron Inputs into the ETC when O₂ Reduction is Suppressed

If fumarate reduction sustains DHODH activity upon inhibition of O₂ reduction, simultaneous loss of both terminal electron acceptors-O₂ and fumarate—will suppress this reaction. Antimycin ablated DHODH activity (as read out by ¹³C₃-UTP production) in SDHB-deficient cells, but had no effect in wild-type cells, knockout cells complemented with the SDHB cDNA, or knockout cells complemented with a class 1 DHODH that directly deposits electrons on fumarate as opposed to UQ (FIG. 3D).

Next, fumarate reduction was tested to determine if it sustains complex I activity in cells incapable of using O₂ as a terminal electron acceptor. Both the NAD+/NADH ratio, which is an indicator of complex I-mediated NADH reoxidation, and the mitochondrial membrane potential (ΔΨ^{Mito}) to which complex I contributes via proton pumping. SDHB-null cells were treated with antimycin, which cannot use fumarate or O₂ as a terminal electron acceptor, had a lower NAD+/NADH ratio than wild-type cells treated with antimycin. The ΔΨ^{Mito}, which was monitored using the fluorescent dye TMRE, was substantially more depolarized in SDHB-null cells after 30 minutes of antimycin treatment than in wild-type cells (FIG. 3E-F), in a fashion complemented by the SDHB cDNA (FIG. 3G). Notably, treatment with 250 nM CCCP, which specifically uncouples the ΔΨ^{Mito} without affecting the plasma membrane potential, reduced fluorescence, indicating that the TMRE dye was not in quench mode. Upon antimycin treatment, wild-type cells displayed an initial reduction in the ΔΨ^{Mito}, which was restored to pre-treatment levels with a similar timing as it takes for antimycin to increase fumarate reduction (FIG. 3F). The effects of antimycin treatment on ΔΨ^{Mito} were further corroborated in the contexts of pharmacologic and genetic suppression of SDHA activity, and importantly, antimycin treatment did not alter MT-DNA copy number in the SDHA and SDHB knockout cells. Thus, fumarate reduction supports partial complex I activity, specifically NADH reoxidation and ΔΨ^{Mito}, in cells incapable of reducing O₂ in the ETC.

To determine if these defects are caused by a lack of electron removal from the ETC onto fumarate and thus an inability to reoxidize UQH₂, the expression of AOX was tested to determine if it could restore DHODH activity and the ΔΨ^{Mito} in cells that are unable to use both fumarate and O₂ as terminal electron acceptors (FIG. 3H). To do so, AOX was expressed in SDHB knockout cells and measured ΔΨ^{Mito} and DHODH activity upon antimycin treatment. Indeed, expression of AOX in SDHB knockout cells almost completely prevented depolarization of ΔΨ^{Mito} and the reduction in DHODH activity upon antimycin treatment (FIG. 3I-J).

An expected consequence of complete loss of electron flow in the ETC is a reduced proliferation rate because the ETC is required for critical biosynthetic pathways like pyrimidine biosynthesis. The proliferation of SDHB-null cells treated with antimycin was less than wild-type cells treated with antimycin in media containing high pyruvate and aspartate. Similarly, treatment of UQCRC2 and COX4 knockout cells with the complex II inhibitor malonic acid significantly reduced their proliferation. Thus, in the context of sufficient metabolic precursors for pyrimidine biosynthesis and the fumarate pool, fumarate reduction can support the proliferation of cells incapable of reducing O₂ in the ETC.

### Example 5. Fumarate is a Terminal Electron Acceptor in Mouse Tissues

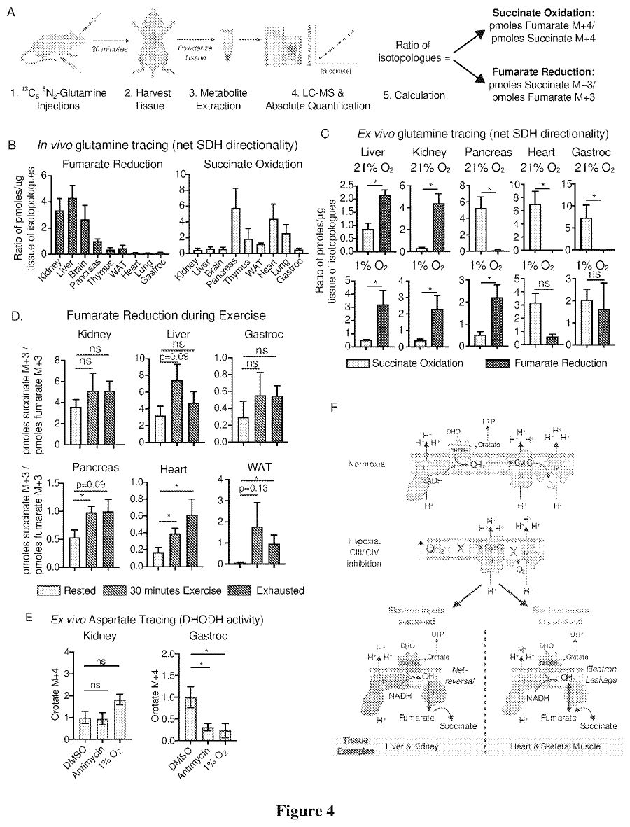

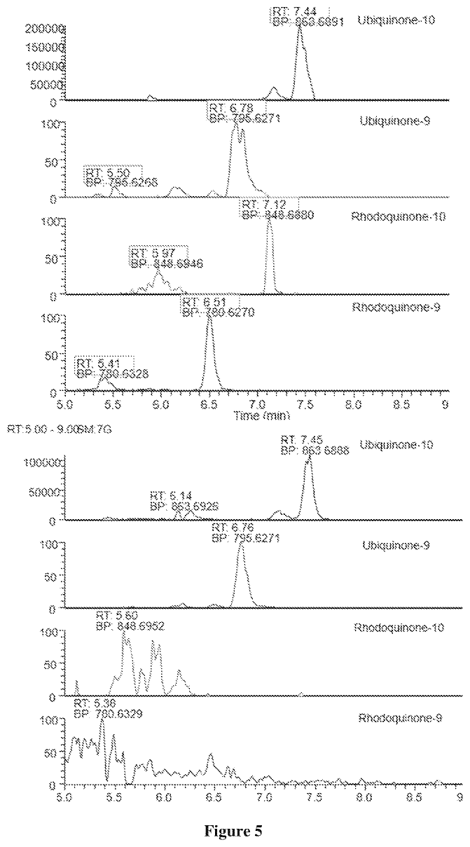

Fumarate reduction was undetectable at 20% O₂, even a reduction to 15% was sufficient to stimulate fumarate reduction, which continued to increase as O₂ levels were lowered until reaching a maximum at 3% O₂. To investigate fumarate reduction in mammalian systems, the ¹³C₅¹⁵N₂-glutamine tracing technique was applied to mouse tissues. Mice were injected with ¹³C₅¹⁵N₂-glutamine, followed by metabolite extraction from tissues, and absolute quantification of the succinate and fumarate isotopologues. SDH activities were quantified by calculating the ratio of the absolute quantification of M+4 isotopologues, representing succinate oxidation, and of M+3 isotopologues, representing fumarate reduction (FIG. 4A). Because these in vivo tracing experiments were performed with a bolus injection, the labeling in the succinate and fumarate pools were not in the steady-state, and therefore, the forward and reverse SDH activities in a given tissue cannot be compared to one another.

The lung, heart, pancreas, thymus, white adipose tissue (WAT), and gastrocnemius muscle catalyzed little to no detectable fumarate reduction and had high levels of succinate oxidation, whereas the kidney, liver, and brain, appeared to catalyze high levels of fumarate reduction (FIG. 4B). The fumarate reduction and succinate oxidation reactions among mouse tissues did not correlate with their total levels or ratios of succinate and fumarate, nor their ratio of UQH₂:UQ. ATP citrate lyase (ACLY), an enzyme required for reductive carboxylation, was low in the heart and high in the liver. Although this positively correlates with their capacity to do fumarate reduction, this correlation did not extend to other tissues. Moreover, all tissues sufficiently enriched the ¹³C₃-fumarate pool upon injection with ¹³C₅¹⁵N₂-glutamine, and across three different time points (10, 20, and 30 minutes) post injection, the ratio of isotopologues representing fumarate reduction and succinate oxidation remained similar, corroborating that interesting differences exist among tissues in their ability to reduce fumarate at physiological O₂ levels.

To determine if the observed labeling in vivo is tissue autonomous, and not from inter-organ transfer of labeled metabolites, ex vivo ¹³C₅¹⁵N₂-glutamine tracing was performed on mouse tissues cultured in incubators set to either 21% or 1% O₂ (FIG. 4C). Ex vivo tracing of ¹³C₅¹⁵N₂-glutamine for at least 16 hours enables the ¹³C₃-fumarate, ¹³C₄-fumarate, ¹³C₃-succinate, and ¹³C₄-succinate isotopologues to be in the steady-state, and thus enables a direct comparison of forward and reverse SDH activities in each tissue. When cultured ex vivo in atmospheric O₂, the liver, kidney, and brain displayed higher levels of fumarate reduction than succinate oxidation, indicating that the SDH complex is intrinsically operating in reverse in these tissues (FIG. 4C). The pancreas, thymus, lung, WAT, heart, and gastrocnemius muscle all favor the succinate oxidation SDH activity over the fumarate reduction SDH activity when cultured in atmospheric O₂ (FIG. 4C). Upon hypoxia exposure, all of these tissues undertook some level of fumarate reduction, but only a subset, including the liver, kidney, brain, pancreas, WAT, thymus, and lung, exhibited net-reversal of SDH, in which the fumarate reduction was greater than succinate oxidation (FIG. 4C). Others, such as the heart and gastrocnemius muscle modestly increased fumarate reduction when exposed to hypoxia, but did not net-reverse the SDH complex (FIG. 4C).

Results from glutamine tracing were corroborated with ¹³C₄-aspartate tracing on mouse tissues cultured ex vivo in 21% and 1% O₂, in which the production of ¹³C₄-succinate was used as a proxy for fumarate reduction when labeling was in the steady-state. Consistent with the apparently constitutive fumarate reduction in the brain, liver, and kidney, and a lack of fumarate reduction in the heart and gastrocnemius muscle, abundant ¹³C₄-succinate labeling was detected in the former but not latter tissues cultured in atmospheric O₂. Upon exposure to hypoxia, ¹³C₄-succinate increased in all tissues, consistent with results from glutamine tracing (FIG. S10C). Interestingly, incorporation of ¹³C₄-aspartate into ¹³C₂-succinate occurred in all tissues except the liver, kidney, and lung, which may be partially driven by differences in citrate synthase levels and oxidative TCA cycle flux among tissues. These data confirm that hypoxia induces fumarate reduction in mouse tissues.

Exercise causes tissue hypoxia, and it has been observed that succinate levels increase in exercising humans and mice. To test if exercise causes an increase in fumarate reduction, mice were challenged to a short (30 minute) or long (90 minute) exercise regimen and then injected them with ¹³C₅¹⁵N₂-glutamine and monitored the ¹³C₃-succinate and ¹³C₃-fumarate isotopologues in the kidney, liver, pancreas, gastrocnemius, heart, and WAT (FIG. 4D, FIG. S11). Upon exercise challenge, ¹³C₃-succinate significantly increased in the gastrocnemius, heart, and WAT, but not in the kidney, liver, or pancreas. To determine if this increase in ¹³C₃-succinate is driven by an increase in fumarate reduction the ratio of the absolute concentration of ¹³C₃-succinate to that of ¹³C₃-fumarate was calculated. These data demonstrate that the pancreas, heart, and WAT, but not the kidney, liver, and gastrocnemius, increase fumarate reduction upon exercise (FIG. 4D).

Because net-reversal of the SDH complex supports DHODH activity in cultured cells when O₂ reduction is blocked (FIG. 3), mouse tissues were tested. DHODH activity was measured in tissues that are capable (the liver and kidney) or incapable (the heart and gastrocnemius muscle) of net-reversing the SDH complex when O₂ reduction is limited. Similar to hypoxia exposure, ex vivo antimycin treatment ablated O₂ consumption in the liver, kidney, heart, and skeletal muscle, but only the liver and kidney maintained net-reversal of the SDH complex. Next, DHODH activity was assessed via ¹³C₄-aspartate incorporation into ¹³C₄-orotate, because labelling of the ¹³C₃-UTP pool was undetectable in most tissues. Consistent with the idea that upon inhibition of O₂ reduction, maintenance of DHODH activity requires SDH reversal, antimycin treatment or hypoxia exposure reduced the levels of ¹³C₄-orotate in the heart and gastrocnemius muscle while they remained unchanged in the liver and kidney (FIG. 4E). These data suggest that the ability to maintain DHODH activity under hypoxia correlates with the ability of a tissue to net-reverse the SDH complex upon inhibition of O₂ reduction.

### Procedures

Cell Culture: The 143B osteosarcoma cell line was cultured in Dulbecco's Modified Eagle Medium (DMEM) (ThermoFisher) supplemented with 5% Heat Inactivated Fetal Bovine Serum (ThermoFisher) and 1% penicillin and streptomycin (ThermoFisher), and 0.1 mg/mL Uridine (Sigma). For experiments that required culturing cells in hypoxia, media was pre-adapted in a hypoxia incubator (Baker Ruskinn) set to the appropriate oxygen level for at least 24 hours. For experiments involving expression of the alternative oxidase (AOX) or the class 1 DHODH, cells in all genetic conditions were treated with 100 ng/mL doxycycline (Takara Bio) for 24 hours to induce its expression. Inhibitors and other supplements: 100 nM-2 μM antimycin (Thermo Fisher Scientific), 5 mM pyruvate (Sigma), 10-20 mM Aspartate (Sigma), 2 μM brequinar (Sigma), 1 μM Rotenone (Sigma), 1 μM-5 μM piericidin (VWR), 10-20 mM Malonic Acid (Sigma), 500 nM Tetramethylrhodamine, Ethyl Ester, Perchlorate (TMRE) (ThermoFisher), 250-500 nM Carbonyl cyanide 3-chlorophenylhydrazone (Sigma). Cells were periodically tested for mycoplasma (SouthernBiotech).

Proliferation: 25,000 cells were seeded in 6-well dishes and counted on day 1 and day 5 on a Beckman Coulter Counter. Doubling times were calculated using the following equation: Doublings=LN(2)/(LN(Day 5 Count/Day 1 Count))

Generation of Stable Cell Lines: Single Guide RNAs (sgRNAs) against SDHA (ACCGTGCATTATAACATGGG) (SEQ ID NO: 1), SDHB (TCCTTTATCACATACATGTG) (SEQ ID NO: 2), UQCRC2 (TAAAGCAGGCAGTAGATATG) (SEQ ID NO: 3), COX4 (GAACTTAATGCGATACACTG) (SEQ ID NO: 4), were subcloned into the plentiCRISPR V1 (Addgene 52963). Subcloned plasmids were co-transfected into HEK293T cells with lentiviral packaging vectors. 143B cells were subsequently infected with the lentivirus and selected with puromycin, generating stable knockout cell lines. Single cells were sorted on a FACS Aria (BD Biosciences) to isolate clones. Clones were cultured and screened for the relevant knockouts. cDNA rescue experiments were performed by expressing cDNAs harboring silent mutations, rendering them resistant to sgRNAs in the knockout cells. cDNAs were cloned into pCW57.1 N-term GFP tTA (Addgene 107551) and stably expressed in knockout cells. Alternative Oxidase cDNA and the class 1 DHODH cDNA were cloned into a pCW57.1-Luciferase backbone (Addgene 9928). All plasmids were sequence verified (Quintara Biosciences) and will be available on Addgene.

Western Blots: Adherent cells were lysed in RIPA buffer (150 mM NaCl, 50 mM Tris-HCl, pH 7.5, 0.1% SDS, 1% Triton-X 100 (Sigma), 0.5% deoxycholate (Sigma), complete EDTA-free protease inhibitor (Sigma)) and clarified by centrifugation. Protein content was quantified using a Pierce BCA Protein Assay Kit (Life Technologies) and 30-50 μg protein were loaded on 12% Tris-Glycine Novex Gels (Invitrogen). Proteins were subsequently transferred to a 0.45 μm PVDF membrane (Sigma). Primary antibodies were used at the following dilutions in 5% BSA:SDHA (Proteintech; 1:1000), NDUFV1 (Abcam; 1:1000), Total OXPHOS Rodent WB Antibody Cocktail-containing UQCRC2, SDHB, and ATP5A (Abcam; 1:1000), COX IV (Cell Signaling Technology; 1:1000), MT-ND5 (Abcam; 1:1000), FLAG (Sigma; 1:1000), HIF1a (Cell Signaling Technology; 1:1000), ATP citrate lyase (Abcam; 1:1000), Citrate Synthase (Cell Signaling; 1:1000). Secondary antibodies were used at the following dilutions in 5% Milk:Anti-rabbit IgG HRP-linked Antibody (1:5000, Cell Signaling Technology), Anti-mouse IgG HRP-linked Antibody (1:5000, Cell Signaling Technology). Blots were developed using SignalFire ECL Reagent (Cell Signaling Technology) and exposed to autoradiography film.

Isolation of Mitochondria for Biochemical Assays: 143B cells were scraped and pelleted at 1000×g for 5 minutes. Cell pellets were washed in 1×PBS and pelleted at 1000×g for 5 minutes. For tissue samples, approximately 100 mg of tissue powder was weighed out. Tissue powder or cells were re-suspended in isolation buffer (200 mM sucrose, 10 mM Tris HCl, 1 mM EGTA/Tris, pH 7.4 (adjusted with 1M HEPES)), transferred into an ice-cold glass homogenizer (VWR), and dounced with 15-30 strokes. Homogenates were transferred into tubes and spun at 600×g for 10 minutes at 4° C. Supernatants were moved to a new tube and centrifugation was repeated. Then, supernatants were moved to a new tube and pelleted at 7,000×g for 10 minutes at 4° C. Pellets were washed in isolation buffer and centrifugation was repeated. Pellets were subsequently stored at −80° C. until use.

Blue Native Gel: Mitochondria were isolated as described above. 50 μg of mitochondrial protein was re-suspended in sample buffer cocktail containing 1× Native PAGE Sample Buffer (Life Technologies) and 8 g digitonin/g mitochondrial protein. Samples were incubated on ice for 20 minutes and then centrifuged at 20,000×g for 10 minutes at 4° C. Coomassie G-250 sample additive (Life Technologies) was combined to ¼th the final protein concentration in the supernatant. Samples were loaded onto NativePAGE 3-12% Bis-Tris Protein Gels (Life Technologies) and initially run in dark blue cathode buffer (1× Native PAGE anode buffer (Life Technologies), 0.2 mg/mL G-250 sample additive (Life Technologies)) for 30 minutes and subsequently switched to light blue cathode buffer (1:10 dilution of dark blue cathode buffer in 1× Native PAGE anode buffer (Life technologies)) for an additional 150 minutes. Gels were transferred onto a 0.45 μm PVDF membrane (Sigma) using 1× NuPAGE Transfer Buffer (Life Technologies) supplemented with 10% methanol. Membranes were subsequently washed with 8% acetic acid followed by 100% methanol and probed for relevant proteins.

Complex 1 activity assay (DCPIP): Purified mitochondria were re-suspended in buffer (0.22 M Mannitol, 0.075 M Sucrose, 1 mM EDTA, 10 mM HEPES pH 7.4, and one complete Protease inhibitor tablet (Sigma)) to a concentration of 5 mg/mL. Mitochondria underwent 5 freeze-thaw cycles to permeabilize them. 50 μg of mitochondrial protein was used for each reaction and mixed with complex 1 activity assay buffer (25 mM KH2PO4 pH 7.5, 1 μM decylubiquinone (Sigma), 300 μM 2,6-dichlorophenolindophenol (VWR), 3.5 g/L BSA) supplemented with 10 μM antimycin (Sigma). Either 1 μM rotenone or equivalent DMSO was added to each well and baseline absorbance at 600 nm was measured on a SpectraMax iD5 plate reader (Molecular Devices). Reactions were initiated by adding NADH (Sigma) to a final concentration of 2 mM and absorbance at 600 nm was monitored over time.

DHODH activity assay: Assay was performed as previously described [3]. Briefly, 1×106 143B cells were seeded 24 hours prior to the assay. Cells were pelleted, resuspended in 1 mL of KPBS (136 mM KCl (Sigma), 10 mM KH2PO4 (Sigma) (pH 7.25)), and permeabilized using 5 freeze-thaw cycles. 280 μL of lysate was incubated with 20 μL of 20 mM dihydroorotate (Sigma) and 700 μL of DHODH activity buffer (500 μM dihydroorotate, 200 mM K2CO3-HCl (pH 8.0), 0.4% Triton X-100 (Sigma), 78 μM decylubiquinone (Sigma)) at 37° C. at 850 rpm in a ThermoMixer (Eppendorf) for 30 minutes. Then, 100 μL of this mixture was incubated with 150 μL of Milli-Q water, 250 μL of 80 mM K2CO3 (Sigma), 250 μL of 8.0 mM K3[Fe(CN)6] (Sigma), and 250 μL of 8.0 mM 4-TFMBAO (Sigma) at 95° C. for 5 minutes. After quenching the reaction in an ice-water bath for 2 minutes, the fluorescence was read in a black Costar 96-well plate with a clear bottom using a SpectraMax iD5 at excitation and emission wavelengths of 320 nm and 420 nm, respectively. Orotate production was quantified using a standard curve in the range of 0-10 mM orotate.

SDH Activity Assay: Purified mitochondria were re-suspended in buffer (0.22 M Mannitol, 0.075 M Sucrose, 1 mM EDTA, 10 mM HEPES pH 7.4, and one complete Protease inhibitor tablet (Sigma)) to a concentration of 5 mg/mL and underwent 5 freeze-thaw cycles to permeabilize them. 30 μg mitochondria (6 uL) were diluted 1:10 (in 54 uL) of SDH activity assay buffer (27.5 mM KH2PO4 pH 7.4, 1.1 mM CoQ-10 (Sigma), 3.5 g/L BSA). Reactions were supplemented with DMSO, 1 μM antimycin, or 1 μM piericidin and pre-incubated at 37° C. for 10 minutes. Fumarate reduction reactions were initiated by adding NADH to a final concentration of 1 mM and fumarate to a final concentration of 10 mM. Succinate oxidation reactions were initiated by adding succinate to a final concentration of 10 mM. Reactions were quenched at the appropriate timepoints with 80% Methanol:20% Water and vortexed for 10 minutes at 4° C. Samples were centrifuged at 16,000×g for 10 minutes at 4° C. and supernatants were dried down and analyzed by LC-MS for succinate and fumarate.

Oxygen Consumption Rate (Seahorse): Respiration was measured on intact 143B cells and on mitochondria purified from tissue on the Seahorse XFe-96 Analyzer (Seahorse Bioscience). Cells were incubated in a non-CO2 37° C. incubator in serum-free Seahorse XF RPMI Media (Seahorse Bioscience, Catalog #103336) supplemented with 5 mM glucose, 2 mM glutamine, and 1 mM pyruvate. Oxygen consumption rate (OCR) was measured over a period of 30 minutes. Oxygen consumption rate was also measured on mitochondria purified from mouse tissues. Mitochondria were incubated in MAS buffer (70 mM sucrose, 220 mM mannitol, 10 mM KH2PO4, 5 mM MgCl2, 2 mM HEPES pH 7.2, 1 mM EGTA, 2% BSA) supplemented with 2 mM ADP, 5 mM glutamate, 5 mM malate, 5 mM pyruvate, and 2.5 mM succinate. Oxygen consumption rate (OCR) was measured over a period of 30 minutes.

Oxygen Consumption Rate (Resipher): 25,000 143B cells were seeded into Thermo Nunc Treated, Flat-Bottom 96-Well Microplate (167008) in standard culture medium 24 hours prior to experimentation. On the day of experimentation, media was changed and the Resipher oxygen sensing lid (Lucid Scientific) was attached. Live oxygen consumption rate was measured for 3 hours and then media was changed to treat cells with the appropriate inhibitors. Live oxygen consumption rate was monitored for an additional 3 hours. Data were analyzed on Resipher web application (Lucid Scientific).

MT-DNA Quantification: Cells were pelleted, washed 1× in PBS, and then lysed in buffer (25 mM NaOH, 0.2 mM EDTA) for 15 minutes at 95° C. Lysis was neutralized with buffer (40 mM Tris-HCl) and centrifuged at 16,000×g for 10 minutes at 4° C. Supernatant containing MT-DNA was quantified on a CFX96 Real-Time System Thermal Cycler (Bio Rad). Primers amplifying the MT-DNA marker D-Loop (Forward Primer-tatcttttggcggtatgcacttttaacag (SEQ ID NO: 5) Reverse Primer—tgatgagattagtagtatgggagtgg) (SEQ ID NO: 6) and the nuclear DNA marker B-Globin (Forward Primer-gtgcacctgactcctgaggaga (SEQ ID NO: 7) Reverse Primer—ccttgataccaacctgcccag) (SEQ ID NO: 8) were used and the relative MT-DNA to nuclear DNA was quantified in each sample [4].

Immunofluorescence assays: 143B cells were seeded onto cover slips at 50% density for 24 hours and then fixed with 4% paraformaldehyde for 15 minutes. Then, cells were permeabilized in 0.25% Triton X-100 in PBS for 5 minutes, followed by blocking in CST blocking buffer (5% Donkey Serum (Sigma), 0.3% Triton X-100) for 30 minutes and incubated for 1 hour with primary antibodies against COX4 (Cell Signaling Technology) or FLAG (Cell Signaling Technology) at 1:200 diluted in buffer (1% BSA, 0.3% Triton X-100 in PBS). Secondary antibodies 568 Donkey anti-rabbit (Invitrogen) and 488 Donkey-anti-mouse (Invitrogen) were diluted to 1:500 in buffer (1% BSA, 0.3% Triton X-100 in PBS) and incubated for an hour. Cover slips were mounted using ProLong Gold+DAPI (ThermoFisher) and imaged on a confocal microscope (Zeiss).

Live Cell Imaging: 143B cells were seeded at 50% density in 6-well dishes 24 hours prior to the experiment. Cells were pre-incubated with 500 nM TMRE dye for 30 minutes and then imaging on a Nikon TE2000 inverted microscope was started. Images were taken at 1 minute intervals over the course of an hour in the mCherry channel while the plate was incubated in a chamber maintaining a temperature of 37° C. and 5% CO2.

Flow Cytometry: Cells were seeded 24 hours prior to the experiment at 50% density. On the day of the experiment, the cells were treated for the designated time with inhibitors. 30 minutes prior to the end of the incubation, 500 nM TMRE dye was added to the wells. Following incubation cells were washed, trypsinized, and filtered through a cell strainer prior to sorting. Fluorescence-activated cell sorting was performed on a LSR-II sorter, in which the fluorescence intensity of individual cells was quantified in the PE channel. Data were analyzed using FlowJo software (TreeStar).

Mouse Experiments: All mouse protocols were approved by the Institutional Animal Care and Use Committee (IACUC) at Massachusetts Institute of Technology. Wild-type C57BL/6J mice acquired from The Jackson Laboratory were housed in the Whitehead Institute Animal Facility and maintained according to the Sabatini Lab protocol approved by the Committee on Animal Care (0718-065-21). In vivo stable isotope tracing experiments were performed by retro-orbital and intraperitoneal bolus injections of ¹³C₅¹⁵N₂-glutamine (50 mg/mL) dissolved in Hank's Balanced Salt Solution (Sigma), 200 μL in each injection site. Mice were euthanized 20 minutes after the injections. Tissue were harvested, flash frozen in liquid nitrogen, and stored at −80° C. until samples were ready for processing.

Exercise on treadmill: 12-week-old female mice were acclimated to a motorized treadmill (TSE systems, incline 5°) for two days prior to the forced running protocol. Mice were trained at 0.17 m/sec on each of the two training days for 20 minutes. On the day of running, mice were subjected to the following running protocol: 0.17 m/sec for 40 min, 0.18 m/sec for 10 min, 0.2 m/sec for 10 min, 0.22 m/sec for 10 min, 0.22-0.25 m/sec for 10 min, 0.25-0.28 m/sec for 10 min, 0.28-0.31 m/sec for 10 min and 0.31-0.32 m/sec for 5 min (Exhausted group). The 30 min group was only subjected to 0.17 m/sec for 30 min. All mice were injected with ¹³C₅¹⁵N₂-glutamine (50 mg/mL), 200 μL intraperitoneal and 50 μL intramuscular, and placed back on the treadmill (Exhausted and 30 min group) or in the cage (Rested group) 15 min before organs were harvested. Organs were harvested and snap frozen immediately after taking the mice off the treadmill.

Stable isotope tracing in cells: Cells were seeded 24 hours prior to the experiment in 6-well dishes at 70% density. For stable isotope tracing of glutamine, glutamine-free DMEM (ThermoFisher) was supplemented with 2 mM ¹³C₅¹⁵N₂-glutamine (Sigma), 1 mM pyruvate, 5% FBS, 1% P/S, 0.1 mg/mL uridine. For stable isotope tracing of aspartate, DMEM containing 5% FBS, and 1% penicillin and streptomycin, and 0.1 mg/mL uridine (Sigma) was supplemented with the designated amount of 13C4-aspartate (Sigma) and the pH adjusted to 7.4. Uridine was left out of the media for stable isotope tracing experiments measuring nucleotide biosynthesis. Unless otherwise stated, for all experiments, cells were treated for 8 hours and metabolites were isolated as described below. Relevant compounds were added at the start of the incubation periods.

Ex vivo stable isotope tracing on tissues: Tissues were excised from mice and washed in 1×PBS. Tissues were then cut into small pieces and placed in the appropriate stable isotope tracing media. Stable isotope tracing media contained either ¹³C₅¹⁵N₂-glutamine (Sigma) or 13C4-aspartate (Sigma) dissolved in DMEM, 5% FBS, 1% P/S. For ex vivo tracing experiments performed on the brain, the same media was used without FBS. Tissues were incubated for the designated time at 37° C. in a normal or hypoxia tissue culture incubator. Tissues were removed from the media, washed with PBS, and flash frozen in liquid nitrogen. Samples could be stored at −80° C. until metabolites were isolated.

Isolation of metabolites from adherent cells: To isolate metabolites, media was removed, cells were washed 2 times with ice cold 1×PBS, and wells were covered in LC-MS grade 80:20 Methanol:Water (ThermoFisher). Plates were scraped on dry ice and lysates were collected into eppendorf tubes. Lysates were vortexed for 10 minutes at 4° C. and centrifuged at 16,000×g for 10 minutes at 4° C. Supernatants were immediately dried down in a Refrigerated CentriVap Benchtop Vacuum Concentrator connected to a CentriVap-105 Cold Trap (Labconco). Dried pellets were stored at −80° C. until they were run on LC-MS.

Isolation of metabolites from tissues: Tissues were flash frozen and powderized with a mortar and pestle in a liquid nitrogen bath. Approximately 30 mg of tissue powder was transferred into eppendorf tubes and re-suspended in 800 uL ice-cold LC-MS grade 60:40 Methanol:Water (ThermoFisher). Samples were vortexed for 10 minutes at 4° C. Then, 500 μL of ice-cold LC-MS grade chloroform (ThermoFisher) was added to the lysate and samples were vortexed for an additional 10 minutes at 4° C. Samples were centrifuged at 16,000×g for 10 minutes at 4° C., creating three layers: the top layer containing polar metabolites, the bottom layer containing non-polar metabolites, and the middle layer containing protein. The top layer was transferred to a new tube, dried down in a speedvac, and subsequently stored at −80° C. until they were analyzed by LC-MS. The protein layer was separated from the non-polar layer and re-suspended in RIPA buffer (150 mM NaCl, 50 mM Tris HCl pH 7.5, 0.1% SDS, 1% Triton-X 100 (Sigma), 0.5% deoxycholate (Sigma), cOmplete EDTA-free protease inhibitor (Sigma)) to isolate protein. Protein in each sample was quantified using the Pierce BCA Protein Assay Kit (Life Technologies). Protein concentrations were used for normalization of sample inputs prior to LC-MS analysis.

Sample prep for LC-MS: Metabolite pellets were re-suspended in LC-MS grade water (ThermoFisher) and vortexed for 10 minutes at 4° C. Samples were centrifuged at 16,000×g for 10 minutes at 4° C. and supernatant was moved into LC-MS vials.

Liquid Chromatography and Mass Spectrometry (polar): A QExactive bench top orbitrap mass spectrometer equipped with an Ion Max source and a HESI II probe coupled to a Dionex UltiMate 3000 HPLC system (Thermo Fisher Scientific) was used to perform all LC-MS experiments. The instrument underwent mass calibration using the standard calibration mixture every 7 days. 2 μL of re-suspended polar metabolite samples were injected onto a SeQuant ZIC-pHILIC 5 μm 150×2.1 mm analytical column equipped with a 2.1×20 mm guard column (MilliporeSigma). The column oven was held at 25° C. and the autosampler tray was held at 4° C. Buffer A was comprised of 20 mM ammonium carbonate, 0.1% ammonium hydroxide. Buffer B was comprised of 100% acetonitrile. The chromatographic gradient was run at a flow rate of 0.150 mL/min as follows: 0-20 min: linear gradient from 80-20% B; 20-20.5 min: linear gradient from 20-80% B; 20.5-28 min: hold at 80% B. The mass spectrometer was operated in full-scan, polarity-switching mode, with the spray voltage set to 3.0 kV, the heated capillary at 275° C., and the HESI probe at 350° C. The sheath gas flow was 40 units, the auxiliary gas flow was 15 units, and the sweep gas flow was 1 unit. MS data was collected in a range of m/z=70-1000. The resolution set at 70,000, the AGC target at 1×106, and the maximum injection time at 20 msec. An additional scan (m/z=220-700) was included in negative mode only to enhance detection of nucleotides.

Ubiquinone and Ubiquinol measurements: Ubiquinone and Ubiquinol were isolated from mitochondria purified by differential centrifugation as described above. Extraction and mass spectrometry protocols were performed with slight modifications to a previously described protocol [5]. Briefly, mitochondria isolated from 25 million cells or approximately 100 mg mouse tissue were resuspended in 500 μL of ethanol and vortexed for 10 minutes at 4° C. Then, 1 mL of hexane was added and the samples were vortexed for an additional 10 minutes at 4° C. Samples were centrifuged at 16,000×g for 10 minutes at 4° C., creating two layers—the top hexane layer and bottom ethanol layer. The top (hexane) fraction was moved into a new tube and dried down. Immediately prior to LC-MS, samples were resuspended in 50 μL of 80:20 ethanol:hexane and loaded into a 4° C. autosampler. 5 μL of samples were injected onto a Luna 3 μm PFP(2) 100 Å, LC Column 100×2 mm. The column oven was held at 25° C. and the autosampler tray was held at 4° C. Buffer A contained water with 0.1% formic acid and Buffer B contained acetonitrile with 0.1% formic acid. The gradient was as follows: 0 to 3 min, hold at 30% A, 3 to 3.25 min, gradient to 2% Buffer A, 3.25 to 5 min, hold at 2% Buffer A, 5 to 6 min, gradient to 1% Buffer A, 6 to 8.75 min, hold at 1% Buffer A, 8.75 to 9 min, gradient to 30% Buffer A and 9 to 10 min, hold 30% Buffer A. The chromatographic gradient was run at a flow rate of 0.50 mL/min. The mass spectrometer was operated in full scan, positive-ion mode, with the spray voltage set to 3.0 kV, the heated capillary at 275° C., and the HESI probe at 350° C. The sheath gas flow was 40 units, the auxiliary gas flow was 15 units, and the sweep gas flow was 1 unit. MS data was collected in a range of m/z=500-1000. The resolution set at 140,000, the AGC target at 3×106, and the maximum injection time at 250 msec.

Data analysis: Metabolites were quantified by integrating peaks using the software TraceFinder 4.1 (ThermoFisher). Metabolites were identified using a 5 ppm mass tolerance, and the expected retention time as determined by an in-house library of chemical standards. 13C and 15N-isotopologues of metabolites were expected to have the same retention time as the 12C and 14N metabolites and were held to the same stringency for mass tolerance (5 ppm). All stable isotope tracing data underwent natural abundance correction using IsoCorrectoR (Bioconductor).

Statistics: Unless otherwise indicated, a two-tailed student's t-test was used to compare the means among experimental groups. All statistical tests had an alpha of 0.05 as the significance threshold. *=P<0.05.

### Example 6. Rhodoquinone is Detected in Mammalian Tissues, Carries Electrons in the ETC, and is Depleted in Microbiome-Free Mice

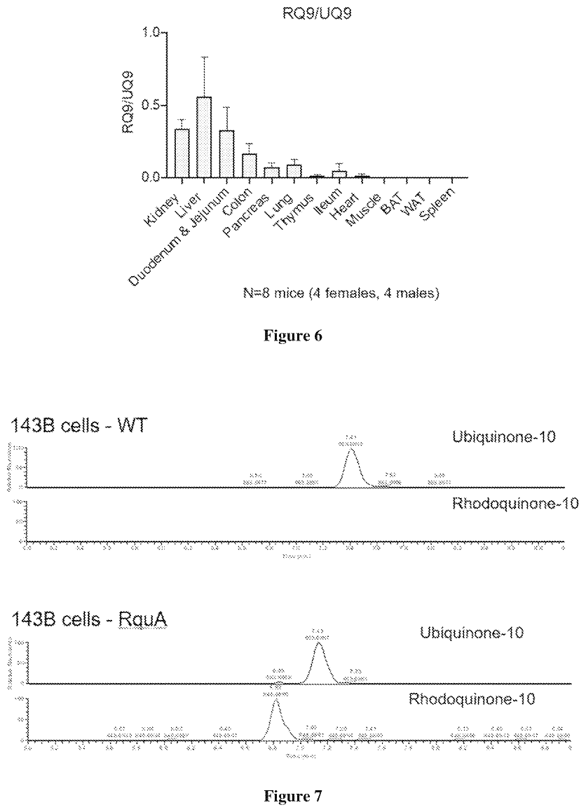

Mitochondria were isolated from mouse tissues and ubiquinone-9 and rhodoquinone-9 were measured by mass spectrometry. Ubiquinone-9 was detected in all mouse tissues. Rhodoquinone-9 was only detected in abundance in mouse kidney, liver, duodenum, jejunum, and colon (FIG. 6). The detection of rhodoquinone was validated using synthesized standards and by fragmentation of rhodoquinone in our standards and in the biological material.

Rhodoquinone-10 was not detected in mitochondria purified from the human 143B cell line cultured in vitro, even though ubiquinone-10 was detected in this sample (FIG. 7). Rhodoquinone-10 can be synthesized in mammalian cells by expressing the enzyme RquA with a mitochondrial localization signal on the N-terminus. RquA converts ubiquinone into rhodoquinone, and when expressed on a doxycycline-inducible promoter, RquA expression leads to a dose-dependent increase in rhodoquinone-10 levels in cultured human 143B cells (FIG. 7).

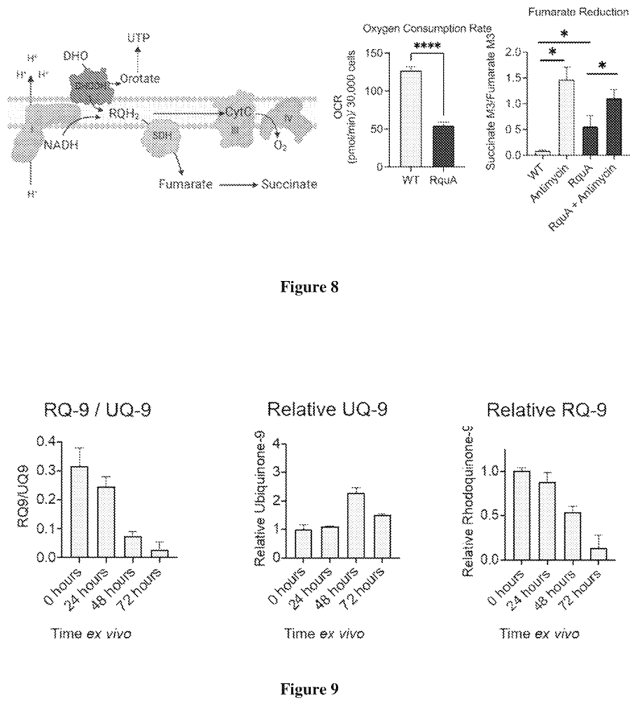

When RquA was expressed in mammalian cells cultured in vitro, the cells had reduced oxygen consumption rate and increased fumarate reduction, indicating that rhodoquinone promotes a circuit of electron flow in the mammalian ETC that diverts electrons away from oxygen as a terminal electron acceptor, and promotes the use of fumarate as a terminal electron acceptor (FIG. 8).

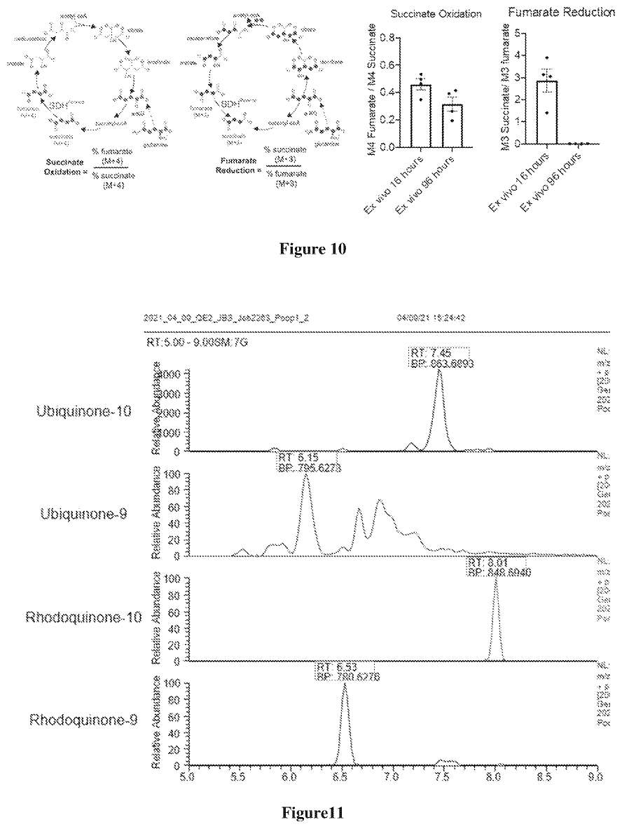

Upon culturing kidney tissue in a petri dish, ex vivo, rhodoquinone-9 levels decreased overtime, while ubiquinone levels remained unchanged (FIG. 9). Rhodoquinone-9 is a previously unknown mammalian metabolite. Without wishing to be bound by any particular theory, it is believed that since the level of rhodoquinone-9 decreases overtime when the kidney is cultured ex vivo, that cultured media is missing a component of the rhodoquinone biosynthetic pathway, and that this component may be coming from the gut microbiome. Culturing kidney ex vivo for 96 hours, which causes a reduction in rhodoquinone levels, leads to a substantial decrease in fumarate reduction without affecting succinate oxidation (FIG. 10), indicating that rhodoquinone presence is required for ubiquitous fumarate reduction in the kidney.

Rhodoquinone-9 was also in mouse feces (FIG. 11). Moreover, the rhodoquinone head group (i.e., rhodoquinone without the isoprene units) was also detected in the feces and mouse tissues that subsequently generate rhodoquinone (FIG. 12), indicating this molecule may be critical for rhodoquinone synthesis in mammalian tissues.

To determine if the microbiome contributes to rhodoquinone biosynthesis in vivo, mitochondria were purified from the kidney of wild-type and germ-free mice. Rhodoquinone-9 was detected in the kidney of wild-type mice. However, the levels were substantially depleted in kidney from germ-free mice (FIG. 13).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating a disease in a subject in need thereof, the method comprising administering to the subject a therapeutically effective amount of a compound of Formula (I): 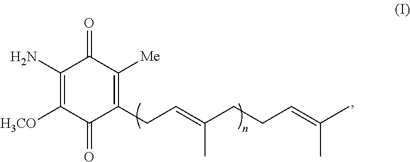
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof;
wherein the disease is a metabolic disorder, hypoxia related disease, or a disease resulting from rhodoquinone depletion.

2. A method of treating a disease resulting from rhodoquinone depletion in a subject in need thereof, the method comprising:
administering to the subject a therapeutically effective amount of a compound of Formula (I): 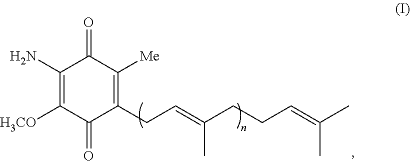
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof;
wherein the subject has previously been administered a statin.

3. A method of treating a disease in a subject in need thereof, the method comprising:
administering to a subject in need thereof an agent or therapy that increases the level of a compound of Formula (I): 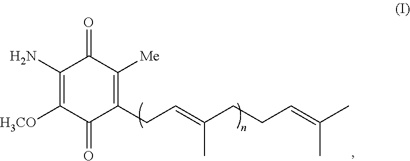
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, in the subject;
wherein the disease is a metabolic disorder, hypoxia related disease, or a disease resulting from rhodoquinone depletion.

4. The method of claim 3, wherein the therapy is a gene therapy.

5. A method of treating a disease in a subject in need thereof, the method comprising administering to the subject a therapeutically effective amount of a compound of Formula (II): 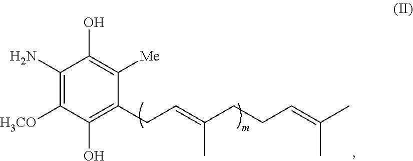
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof;
wherein the disease is a metabolic disorder, hypoxia related disease, or a disease resulting from rhodoquinone depletion.

6. A method of treating a disease resulting from rhodoquinone depletion in a subject in need thereof, the method comprising:
administering to the subject a therapeutically effective amount of a compound of Formula (II): 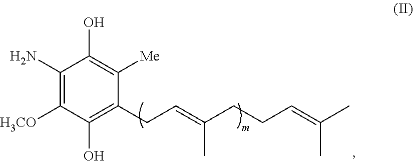
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof;
wherein the subject has previously been administered a statin.

7. A method of treating a disease in a subject in need thereof, the method comprising:
administering to a subject in need thereof an agent or therapy that increases the level of a compound of Formula (II): 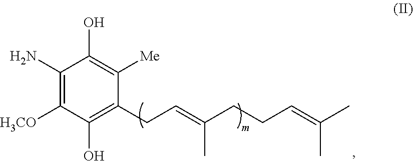
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; in the subject;
wherein the disease is a metabolic disorder, hypoxia related disease, or a disease resulting from rhodoquinone depletion.

8. The method of claim 7, wherein the therapy is a gene therapy.

9. The method of any one of claims 1-4, wherein n is 8, 9, 10, or 11.

10. The method of any one of claims 1-4, wherein n is 9 or 10.

11. The method of any one of claims 1-4, wherein n is 9.

12. The method of any one of claims 1-4, wherein n is 10.

13. The method of any one of claims 5-8, wherein m is 8, 9, 10, or 11.

14. The method of any one of claims 5-8, wherein m is 9 or 10.

15. The method of any one of claims 5-8, wherein m is 9.

16. The method of any one of claims 5-8, wherein m is 10.

17. The method of any one of claims 1-16, wherein the subject is a mammal.

18. The method of any one of claims 1-17, wherein the subject is a human.

19. The method of any one of claims 1-18, wherein the disease is a metabolic disorder.

20. The method of any one of claims 1-19, wherein the disease is obesity.

21. The method of any one of claims 1-19, wherein the disease is diabetes.

22. The method of any one of claims 1-18, wherein the disease is ischemia.

23. The method of any one of claims 1-18, or 22, wherein the ischemia is ischemia of the pancreas, adipose tissue, skeletal muscle, brain, kidney, liver, gastrointestinal tract, heart, or lung.

24. The method of any one of claims 1-18, or 22-23, wherein the ischemia is ischemia of the pancreas.

25. The method of any one of claims 1-18, or 22-23, wherein the ischemia is ischemia of the adipose tissue.

26. The method of any one of claims 1-18, or 22-23, wherein the ischemia is ischemia of the skeletal muscle.

27. The method of any one of claims 1-18, or 22-23, wherein the ischemia is ischemia of the brain.

28. The method of any one of claims 1-18, or 22-23, wherein the ischemia is ischemia of the kidney.

29. The method of any one of claims 1-18, or 22-23, wherein the ischemia is ischemia of the liver.

30. The method of any one of claims 1-18, or 22-23, wherein the ischemia is ischemia of the gastrointestinal tract.

31. The method of any one of claims 1-18, or 22-23, wherein the ischemia is ischemia of the heart.

32. The method of any one of claims 1-18, or 22-23, wherein the ischemia is ischemia of the lung.

33. The method of any one of claims 1-18, wherein the disease is hypoxia.

34. The method of any one of claims 1-18, or 33, wherein the disease is exercise-induced hypoxia.

35. The method of any one of claims 1-18, or 33, wherein the disease is hypoxia resulting from ischemia.

36. The method of any one of claims 1-18, or 33-35, wherein the hypoxia is hypoxia of the heart.

37. The method of any one of claims 1-18, or 33-35, wherein the hypoxia is hypoxia of the lung.

38. The method of any one of claims 1-18, wherein the disease is oxidative stress.

39. The method of any one of claims 1-18, wherein the disease is a neuromuscular disorder.

40. The method of any one of claims 1-18, or 39, wherein the neuromuscular disorder is a mitochondrial myopathy.

41. The method of any one of claims 1-18, wherein the disease is a neurodegenerative disorder.

42. The method of any one of claims 1-18, or 41, wherein the neurodegenerative disorder results from mutations in the mitochondrial DNA.

43. The method of any one of claims 1-18, wherein the disease is CoQ10 deficiency.

44. The method of any one of claims 1-18, wherein the disease is mitochondrial complex 3 deficiency.

45. The method of any one of claims 1-18, wherein the disease is mitochondrial complex 4 deficiency.

46. A composition comprising a therapeutically effective amount of a compound of Formula (I): 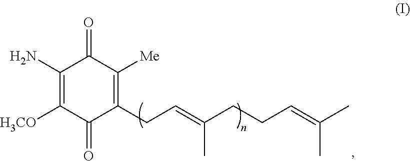
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, and an additional pharmaceutical agent.

47. The composition of claim 46, wherein n=8 or 9.

48. A composition comprising a therapeutically effective amount of a compound of Formula (II): 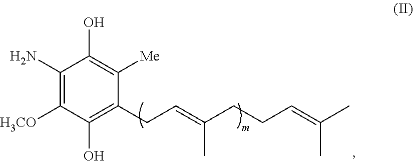
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, and an additional pharmaceutical agent.

49. The composition of claim 48, wherein m=8 or 9.

50. The composition of any one of claims 46-49, wherein the additional pharmaceutical agent is an anti-obesity agent.

51. The composition of any one of claims 46-49, wherein the additional pharmaceutical agent is a probiotic.

52. The composition of any one of claims 46-49, wherein the additional pharmaceutical agent is an antibiotic.

53. The composition of any one of claims 46-49, wherein the additional pharmaceutical agent is a statin.

54. A compound of Formula (I): 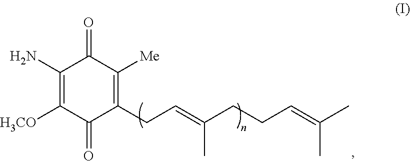
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in treating a disease in a subject in need thereof;
wherein the disease is a metabolic disorder, hypoxia related disease, or a disease resulting from rhodoquinone depletion.

55. A compound of Formula (I): 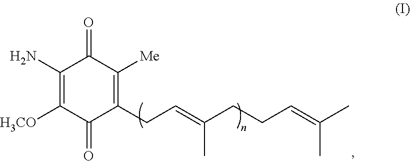
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in the manufacture of a medicament for the treatment of a disease in a subject in need thereof;
wherein the disease is a metabolic disorder, hypoxia related disease, or a disease resulting from rhodoquinone depletion.

56. Use of a compound of Formula (I): 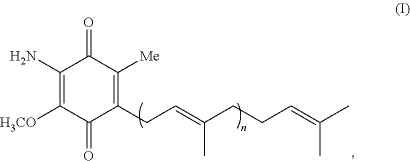
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for treating a disease in a subject in need thereof;
wherein the disease is a metabolic disorder, hypoxia related disease, or a disease resulting from rhodoquinone depletion.

57. A compound of Formula (II): 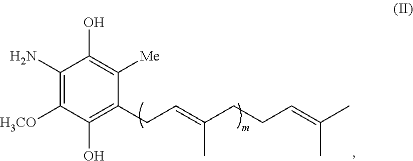
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in treating a disease in a subject in need thereof;
wherein the disease is a metabolic disorder, hypoxia related disease, or a disease resulting from rhodoquinone depletion.

58. A compound of Formula (II): 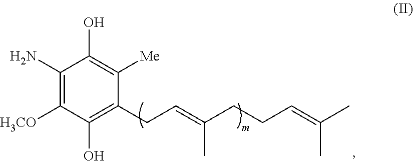
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for use in the manufacture of a medicament for the treatment of a disease in a subject in need thereof;
wherein the disease is a metabolic disorder, hypoxia related disease, or a disease resulting from rhodoquinone depletion.

59. Use of a compound of Formula (II): 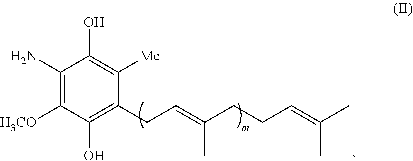
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof, for treating a disease in a subject in need thereof;
wherein the disease is a metabolic disorder, hypoxia related disease, or a disease resulting from rhodoquinone depletion.

60. The compound or use of any one of claims 54-56, wherein n is 9 or 10.

61. The compound or use of any one of claims 54-56, wherein n is 9.

62. The compound or use of any one of claims 54-56, wherein n is 10.

63. The compound or use of any one of claims 57-59, wherein m is 9 or 10.

64. The compound or use of any one of claims 57-59, wherein m is 9.

65. The compound or use of any one of claims 57-59, wherein m is 10.

66. The compound or use of any one of claims 54-65, wherein the disease is a metabolic disorder.

67. The compound or use of any one of claims 54-66, wherein the disease is obesity.

68. The compound or use of any one of claims 54-65, wherein the disease is ischemia.

69. The compound or use of any one of claims 54-65, or 68, wherein the ischemia is ischemia of the kidney, liver, gastrointestinal tract, heart, or lung.

70. The compound or use of any one of claims 54-65, or 68-69, wherein the ischemia is ischemia of the heart.

71. The compound or use of any one of claims 54-65, or 68-69, wherein the ischemia is ischemia of the lung.

72. The compound or use of any one of claims 54-65, wherein the disease is hypoxia.

73. The compound or use of any one of claims 54-65, or 72, wherein the hypoxia is hypoxia of the kidney, liver, gastrointestinal tract, heart, or lung.

74. The compound or use of any one of claims 54-65, or 72-73, wherein the hypoxia is hypoxia of the heart.

75. The compound or use of any one of claims 54-65, or 72-73, wherein the hypoxia is hypoxia of the lung.

76. The compound or use of any one of claims 54-65, or 72-75, wherein the disease is exercise-induced hypoxia.

77. The compound or use of any one of claims 54-65, or 72-76, wherein the disease is hypoxia resulting from ischemia.

78. The compound or use of any one of claims 54-77, wherein the disease is oxidative stress.

79. The compound or use of any one of claims 54-65, wherein the disease is a neuromuscular disorder.

80. The compound or use of any one of claims 54-65, or 79 wherein the neuromuscular disorder is a mitochondrial myopathy.

81. The compound or use of any one of claims 54-65, wherein the disease is a neurodegenerative disorder.

82. The compound or use of any one of claims 54-65, or 81, wherein the neurodegenerative disorder results from mutations in the mitochondrial DNA.

83. The compound or use of any one of claims 54-65, wherein the disease is CoQ10 deficiency.

84. The compound or use of any one of claims 54-65, wherein the disease is mitochondrial complex 3 deficiency.

85. The compound or use of any one of claims 54-65, wherein the disease is mitochondrial complex 4 deficiency.

86. A composition comprising a compound of any one of claims 54-85; and
an additional pharmaceutically acceptable excipient.

87. A kit comprising:
a compound of Formula (I): 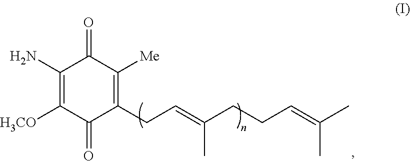
wherein n=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; and
instructions for using the compound, or a pharmaceutically acceptable salt, solvate, hydrate, polymorph, co-crystal, tautomer, stereoisomer, isotopically labeled derivative, or prodrug thereof.

88. A kit comprising:
a compound of Formula (II): 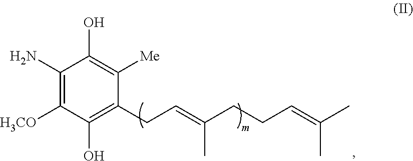
wherein m=1, 2, 3, 4, 5, 6, 7, 8, 9, 10, 11, or 12; or a pharmaceutically acceptable salt, solvate, hydrate, polymorph, co-crystal, tautomer, stereoisomer, isotopically labeled derivative, or prodrug thereof; and
instructions for using the compound, or a pharmaceutically acceptable salt, solvate, hydrate, polymorph, co-crystal, tautomer, stereoisomer, isotopically labeled derivative, or prodrug thereof.

89. The kit of claim 87 or 88, further comprising an additional pharmaceutical agent.

90. The composition or kit of any one of claims 87-89, wherein the additional pharmaceutical agent is an anti-obesity agent.

91. The composition or kit of any one of claims 87-89, wherein the additional pharmaceutical agent is a probiotic.

92. The composition or kit of any one of claims 87-89, wherein the additional pharmaceutical agent is an antibiotic.

93. The composition or kit of any one of claims 87-89, wherein the additional pharmaceutical agent is a statin.

94. The composition or kit of any one of claims 87-89, wherein the additional pharmaceutical agent is a plasmid.


SEQUENCE LISTING (8 sequences)

SEQ ID NO: 1 - DNA, 20 bp, synthetic construct
  source 1..20: mol_type = other DNA
accgtgcattataacatggg

SEQ ID NO: 2 - DNA, 20 bp, synthetic construct
  source 1..20: mol_type = other DNA
tcctttatcacatacatgtg

SEQ ID NO: 3 - DNA, 20 bp, synthetic construct
  source 1..20: mol_type = other DNA
taaagcaggcagtagatatg

SEQ ID NO: 4 - DNA, 20 bp, synthetic construct
  source 1..20: mol_type = other DNA
gaacttaatgcgatacactg

SEQ ID NO: 5 - DNA, 29 bp, synthetic construct
  source 1..29: mol_type = other DNA
tatcttttggcggtatgcacttttaacag

SEQ ID NO: 6 - DNA, 26 bp, synthetic construct
  source 1..26: mol_type = other DNA
tgatgagattagtagtatgggagtgg

SEQ ID NO: 7 - DNA, 22 bp, synthetic construct
  source 1..22: mol_type = other DNA
gtgcacctgactcctgaggaga

SEQ ID NO: 8 - DNA, 21 bp, synthetic construct
  source 1..21: mol_type = other DNA
ccttgataccaacctgcccag